(19) United States
(12) Patent Application Publication
(10) Pub. No.: US 2003/0077607 A1
(43) Pub. Date: Apr. 24, 2003

(54) Methods and tools for nucleic acid sequence analysis, selection, and generation

(76) Inventors: Anton J. Hopfinger, Lake Forest, IL (US); Peter V. Riccelli, Tinley Park, IL (US); Petr Pancoska, Evanston, IL (US); Albert S. Benight, Schaumburg, IL (US)

Correspondence Address: LOUIS MYERS, Fish & Richardson P.C., 225 Franklin Street, Boston, MA, 02110-2804, US

(21) Appl. No.: 10/095,923

(22) Filed: Mar. 11, 2002

Related U.S. Application Data
(60) Provisional application No. 60/274,598, filed on Mar. 10, 2001.

(51) Int. Cl. C12Q 1/68; G06F 19/00; G01N 33/48; G01N 33/50

(52) U.S. Cl. 435/6; 702/20

(57) ABSTRACT

The present invention provides methods and means for analyzing, designing, selecting and generating oligomer sequences, such as those for use in multiplex array-based nucleic acid probe systems, down to the selection of a single pair of optimal primer/target oligomers. Sequences are represented by a function of sequence context, called the context functional descriptor. In addition to the consideration of base pairing and nearest-neighbor analysis, the present computational methods incorporate the use of context functional descriptors and correlation matrices to account for higher-order thermodynamic interactions between nucleic acid sequences.

### RELATED APPLICATIONS

This application claims priority to U.S. provisional application No. 60/274,598 filed on Mar. 10, 2001, the contents of which are incorporated herein by reference.

### TECHNICAL FIELD

This invention relates to the field of bioinformatics, and more particularly to the analysis, selection, and generation of nucleic acid sequences which, for example, can be used for microarray applications involving nucleic acid hybridization.

### BACKGROUND

Large-scale, high throughput, combinatorial approaches to nucleic acid analysis are emerging as powerful tools for widespread applications in detecting, discriminating and analyzing large numbers of DNA sequences via multiplex hybridization schemes. Duggan, D. J., et al., “Expression profiling using cDNA microarrays” Nature Genet. Suppl., 21:10-14 (1999); Lipshutz, R. J., et al. “Using oligonucleotide probe arrays to access genetic diversity”, Biotechniques, 19:442-447 (1995); O'Donnell-Maloney, M. J., et al. “The development of microfabricated arrays for DNA sequencing and analysis,” Trends In Biotechnol., 14:401-407 (1996); de Saizieu, A., et al. “Bacterial transcript imaging by hybridization of total RNA to oligonucleotides arrays,” Nature Biotechnol., 16:45-48 (1998); Southern, E. M, Mir, K and Shchepinov, M. “Molecular interactions on microarrays,” Nat. Genet. Suppl., 21:5-9 (1999); Chen, J. J., et al., “Profiling expression patterns and isolating differentially expressed genes by cDNA microarray system with colorimetry detection,” Genomics, 51:313-324 (1996).

Microarray technologies offer the benefit of being high throughput and the potential of being exquisitely accurate. Cheung, V. G., et al., “Making and reading microarrays,” Nat. Genet., 21:15-19 (1999); Schena, M., et al., “Quantitative monitoring of gene expression patterns with a complementary DNA microarray,” Science, 270:467-470 (1995); Ramsay, G., “DNA chips: state of the art,” Nat. Biotechnol., 16:40-44 (1998); Shalon, D., et al., “A DNA microarray system for analyzing complex DNA samples using two-color fluorescent probe hybridization,” Genome Res., 6:639-645 (1996); Khan, J., et al., “Expression profiling in cancer using cDNA microarrays,” Electrophoresis, 20:223-229 (1999); Szalli, Z. et al., “Genetic network analysis in light of massively parallel biological data acquisition,” Pac. Symp. Biocomput., 1999:5-16 (1999); Chee, M., et al., “Accessing genetic information with high-density DNA arrays,” Science, 274:610-614 (1996); Pease, A. C., et al., “Light-generated oligonucleotide arrays for rapid DNA sequence analysis,” Proc. Natl. Acad. Sci. USA, 91:5022-5026 (1994); Fodor, S. P., et al., “Multiplexed biochemical assays with biological chips,” Nature, 364:555-556 (1993); Shoemaker, D. D., et al., “Quantitative phenotypic analysis of yeast deletion mutants using a highly parallel molecular bar-coding strategy,” Nat. Genet., 14:450-456 (1996); Eisen, M. B., et al., “Cluster analysis and display of genome-wide expression patterns,” Proc. Natl. Acad. Sci., USA, 95:14863-14868 (1998).

However, the best results are obtained when sequences on the microarrays are appropriately designed such that hybridization occurs with high fidelity in the intended sequence-specific manner. Microarray based assays are now finding many uses, and have enormously high expectations for applications in many facets of molecular biology research and nucleic acids diagnostics. DeRisi, J., et al., “Use of a cDNA microarray to analyse gene expression patterns in human cancer,” Nat. Genet. (14) p. 457-460 (1996). Methods and strategies for analyzing genomic sequences are expected to increasingly employ microarray-based approaches. Forozan, F., et al., “Genome screening by comparative genomic hybridization,” Trends Genet. 13:405-409 (1997); McKenzie, S. E., et al., “Parallel molecular genetic analysis,” Eur. J. Hum. Genet. 6:417-429 (1998). Great benefits for human health and life quality are obtainable when diagnosis or treatment is targeted specifically based on an individual's genotype. Schena, M., et al., “Microarrays: biotechnology's discovery platform for functional genomics,” Trends Biotechnol., 16:301-306 (1998).

Realizing these goals will be more likely if microarray technologies are perfected to their optimum capabilities. For microarrays to be reliable, accessible and affordable and fulfill market expectations, superior sequence design strategies are required.

In general, nucleic acid microarrays are of two different classes, cDNA and oligonucleotide arrays. The probes on the surface in each case differ significantly in length. cDNA microarrays are made by attaching prepared libraries of cDNA probes to a microarray surface. These cDNA probes generally range from approximately 300 to 900 bases (more or less). Oligonucleotide arrays have short synthetically prepared oligonucleotide probes that vary in length from approximately 15 to 70 bases. Oligonucleotide arrays can be high density (over 100,000 probe sites on a single surface) prepared by in situ synthesis using photolithography techniques in combination with laser induced photo activated reagents (Affymetrix). Alternatively, oligonucleotide arrays can be of mid or low density (about 5,000 or fewer probe sites on a surface) prepared by various spotting methods. Oligonucleotide array spotting methods are known in the art, as used by companies such as Incyte, Hyseq, Agilent, GPC Biotech, Genosys Biotechnologies, Compugen, Clontech, Corning Inc., Operaon (Qiagen), Genomic Solutions, Genometrix, NEN Life Sciences, Protogen Laboratories, and Research Genetics. Oligonucleotide microarrays can be of the universal or specific type. Gerry, N. P., et al., “Universal DNA microarray method for multiplex detection of low abundance point mutations,” J. Mol. Biol., 292:251-62 (1999).

Many platforms utilize solid-support bound oligonucleotide probes to hybridize and thereby capture single-stranded targets. The majority of formats commonly employ linear single-stranded oligonucleotide probes on two-dimensional surfaces (glass slides, microtiter plates, gel pads for example), but bead-based formats are also emerging (Luminex). Regardless of the format, hybridization by nucleic acid targets to tethered oligonucleotide probes is the central event in the detection of nucleic acids on microarrays. Sequence design based on target sequences and their sequence-dependent stability is essential to achieve optimum hybridization performance.

Designing sequences for multiplex reactions requires consideration of two aspects of sequences. These are referred to as the informatics and engineering aspects. The informatics component of sequence design concerns the process of defining sequences uniquely diagnostic of the desired targets. Much attention has been paid to this aspect and a number of methods and companies tout “better” sequence screening and selection capabilities for identifying unique target sequences. Lee M., K. et al., “SeqHelp: a program to analyze molecular sequences utilizing common computational resources,” Genome Res. 8:306-312 (1998); Zhang M. Q. “Large-scale gene expression data analysis: a new challenge to computational biologists,” Genome Res, 8:681-688 (1999); Buck G. A., et al., “Design strategies and performance of custom DNA sequencing primers,” Biotechniques, 27:528-36 (1999); Talaat A. M. et al., “Genome-directed primers for selective labeling of bacterial transcripts for DNA microarray analysis,” Nat. Biotechnol. 6:679-682 (2000).

The engineering aspect of sequence design involves the selection of sequences that display consistent and dependable hybridization characteristics, such as uniform signal intensity, comparable thermostability and zero cross-hybridization with other strands.

There is a need for better nucleic acid selection methods to address the engineering aspect of sequence design. In particular, there is a need for a more accurate method of analyzing and predicting nucleic acid hybridization, including methods of analyzing and predicting cross-hybridization involving two nucleic acid sequences that are not perfectly complementary. Such methods will find use, for example, in the selection of nucleic acid probes that serve the needs of today's large-scale, high-throughput commercial applications. The present invention provides such methods and tools, which are based on analytical models that incorporate the effects of sequence context into the analysis of the stability and thermodynamic properties of nucleic acid sequences, e.g., pairs of nucleic acid sequences.

### SUMMARY

The sequence analysis, selection, and generation capabilities of the technology enabled by the methods described herein are applicable to problems associated with the engineering aspects of sequence design. In particular, the present invention considers effects of sequence context on nucleic acid hybridization. The influence of sequence context on hybridization has not generally been considered in sequence design strategies of the prior art. Methods of the invention use classical thermodynamic treatments of sequence dependent stability that are augmented by considerations of thermodynamic contributions from sequence context. The methods of the invention can be used to select sets of sequences having defined hybridization properties and display optimal performance when used for microarray or other types of applications involving high fidelity nucleic acid hybridization. For example, a set of such sequences can exhibit isothermal hybridization properties, e.g., the melting temperature for each sequence in the set (when bound to its perfect complement) can fall within a narrow temperature interval (e.g., 4° C.), and each sequence in the set can be non-cross hybridizing with the complements of the other members of the set.

Accordingly, in one aspect, the invention features a method of analyzing a nucleic acid duplex. The method includes:

constructing a CFD describing the interaction of a first nucleic acid sequence with a second nucleic acid sequence, e.g., a perfect complement of the first nucleic acid sequence or a nucleic acid sequence other than the perfect complement,

thereby analyzing a nucleic acid duplex.

In a preferred embodiment, the second nucleic acid sequence is a perfect complement of the first nucleic acid sequence. In other preferred embodiments, the second nucleic acid sequence differs, e.g., by one or more bases, from the perfect complement of the first nucleic acid sequence.

In preferred embodiments, construction of the CFD involves the analysis of many different interaction states for the first and second nucleic acid sequences, wherein the interaction states are identified by sliding the first nucleic acid sequence over the second nucleic acid sequence, one base at a time, and each new position is considered an interaction state, e.g., as described herein. In other preferred embodiments, construction of the CFD allows for shifted base-pairing contributions to the measured stability of states wherein there are at least two sequential base-pair mismatches, e.g., as discussed in Example 2.

In preferred embodiments, the CFD contains at least N+M-1 data points, wherein N is the length of the first nucleic acid sequence and M is the length of the second nucleic acid sequence. In other preferred embodiments, the CFD contains data points that are predictions of thermodynamic values, e.g., ΔH, ΔG, ΔS or some combination thereof, corresponding to the different interaction states of the first and second nucleic acid sequences. In other embodiments, the CFD contains data points that are predictions of the tₘ associated with the different interaction states of the first and second nucleic acid sequences.

In preferred embodiments, the first nucleic acid sequence has a length N, wherein N is about 5 to 50, 10 to 40, or about 15 to 200, 20 to 100, or preferably 25 to 75 bases in length. In other preferred embodiments, the second nucleic acid sequence is the same length as the first nucleic acid sequence. In other embodiments, the second nucleic acid sequence has a length that differs from that of the first nucleic acid sequence.

Any nucleic acid sequence can be analyzed using these methods, including natural nucleic acid sequences of fragments thereof, synthetic nucleic acid sequences, and nucleic acid sequences that have been pre-selected, e.g., unique targeting sequences.

In another aspect, the invention features a method of identifying a CFD component associated with a property of a nucleic acid sequence or a peptide encoded by one strand of the nucleic acid duplex. The method includes:

optionally, providing CFDs for a training set of nucleic acid sequences and their perfect complements;

identifying one or more components of each of the CFDs, either directly or, e.g., by principal component analysis, partial least squares analysis, Fourier analysis, or any method for the decomposition of sets of functions into unique sets of components;

identifying a component, the presence, value, or contribution of which is correlated, either negatively or positively, with a property of the nucleic acid sequences of the training set or peptides encoded thereof, thereby identifying a CFD component associated with a property of a nucleic acid sequence or peptide encoded by the nucleic acid sequence.

In preferred embodiments, the property can be a thermodynamic, e.g., ΔH, ΔG, ΔS or some combination thereof, or related property, e.g., tₘ, associated with the interaction of a nucleic acid sequence and its perfect complement. In another embodiment, the property can be the ability of the nucleic acid to interact with another molecule, e.g., a protein (e.g., a transcription factor, a histone, or a ribosomal protein), another nucleic acid molecule, or a chemical compound.

In preferred embodiments, identifying a component that is correlated, either negatively or positively, with a property of the nucleic acid sequences of the training set involves principle component analysis. In other preferred embodiments, identifying a component that is correlated, either negatively or positively, with a property of the nucleic acid sequences of the training set involves the use and training of a neural network.

Any nucleic acid sequence can be analyzed using these methods, including natural nucleic acid sequences of fragments thereof, synthetic nucleic acid sequences, and nucleic acid sequences that have been pre-selected, e.g., unique targeting sequences.

In another aspect, the invention features, a method of analyzing a sample nucleic acid sequence. The method includes:

providing a CFD for the sample nucleic acid sequence and its perfect complement;

identifying one or more components of each of the CFDs, either directly or, e.g., by principal component analysis, partial least squares analysis, Fourier analysis, or any method for the decomposition of sets of functions into unique sets of components; and

determining if a pre-selected component, known to be present in, associated with, or a contributor to the CFDs of nucleic acid sequences having a particular property, is present as a component in the CFD;

thereby analyzing the nucleic acid.

In a preferred embodiment, the association of the CFD component with the property was determined by a method disclosed herein, e.g., a method involving principal component analysis, partial least squares analysis, Fourier analysis, or any method for the decomposition of sets of functions (e.g., CFDs) into unique sets of components, and the correlation of CFD components with observed properties of the sequences represented by the CFDs.

In preferred embodiments, the property can be a thermodynamic, e.g., ΔH, ΔG, ΔS or some combination thereof, or related property, e.g., tₘ, associated with the interaction of a nucleic acid sequence and its perfect complement. In another embodiment, the property can be the ability of the nucleic acid to interact with another molecule, e.g., a protein (e.g., a transcription factor, a histone, or a ribosomal protein), another nucleic acid molecule, or a chemical compound.

Any nucleic acid sequence can be analyzed using these methods, including natural nucleic acid sequences of fragments thereof, synthetic nucleic acid sequences, and nucleic acid sequences that have been pre-selected, e.g., unique targeting sequences.

In another aspect, the invention features methods of comparing nucleic acid sequences. The methods include:

providing a first CFD for a first nucleic acid sequence and its perfect complement;

providing a second CFD for a second nucleic acid sequence and its perfect complement; and

comparing the first and second CFDs, e.g., in a quantitative manner, e.g., by calculating a correlation coefficient, the variance, or some other statistical measure of similarity.

In preferred embodiments, the first and second CFDs contain data points that are predictions of thermodynamic values, e.g., ΔH, ΔG, ΔS or some combination thereof, corresponding to the different interaction states of the nucleic acid sequences with their respective perfect complements. In other embodiments, the first and second CFDs contain data points that are predictions of the tₘ associated with the different interaction states of the nucleic acid sequences with their respective perfect complements. In a preferred embodiment the calculation of CFD includes accounting for loop structures inferred from mismatches, e.g., more than a selected number of mismatches occurring within a selected distance of one another are considered a loop and matches within that loop are included, though discounted, in the determination of the parameter value provided at that position.

In preferred embodiments, the minima or maxima of the first and second CFDs are aligned prior to being compared. In preferred embodiments, the comparison involves the calculation of a correlation coefficient for the first and second CFDs. In other preferred embodiments, the comparison involves a calculation of the variance between the first and second CFDs. In still other preferred embodiments, the comparison involves the calculation of more than one measure of similarity. For example, the comparison can include a correlation coefficient, a measure of variance, a value for the amount of nucleotide overlap that occurs upon alignment of the CFDs, e.g., when the CFDs are aligned on absolute minima or maxima for a parameter, the difference in AH of the absolute minima of the CFDs, as well as the number of mismatches that occur in a particular state.

In some embodiments, providing the first and second CFDs involves constructing the CFDs, e.g., as described herein. In other embodiments, the CFDs are stored in a database and are simply retrieved for the purpose of comparison.

In preferred embodiments, statistically similar CFDs provide an indication that the corresponding nucleic acid duplexes have similar properties, e.g., thermodynamic properties, e.g., tₘ's or the ability of the duplexes to interact with other molecules, e.g., proteins (e.g., transcription factors, histones, or ribosomal proteins), other nucleic acid molecules, or chemical compounds.

In other embodiments, the methods of the invention further include:

constructing a first CFD for a first nucleic acid sequence and its perfect complement;

constructing a second CFD for a second nucleic acid sequence and its perfect complement;

comparing the first and second CFDs, e.g., in a quantitative manner, e.g., by calculating a correlation coefficient, the variance, or some other statistical measure of similarity; and, optionally

recording the first CFD, the second CFD, a measure of the similarity of the first and second CFDs, or some combination thereof, e.g., in a database.

In some preferred embodiments, the methods can be extended to the analysis of a set t of nucleic acid sequences. For example, the methods can be applied to scanning a set of nucleic acid sequences, e.g., taken from a gene or genome, and identifying those sequences that are most similar or most dissimilar. The methods include the following steps:

defining the desired length, N, e.g., between about 15 to 200, 20 to 100, or preferably 25 to 75 bases, of the nucleic acid sequences that will be compared with one another;

generating a set of sequences of length N that will be compared with one another by starting at the first base of the sequence of interest, e.g., a gene or genome, and moving a window of length N over the sequence of interest, one base at a time, so as to identify a resulting set consisting of all contiguous sequences of length N contained within the sequence of interest (typically about L-N, where L is the length, in bases, of the sequence of interest);

generating CFDs for each sequence in the resulting set of sequences and its perfect complement; and

determining the similarity between all combinations of the CFDs, e.g., by calculating correlation coefficients, variance, or some other statistical measure of similarity for all combindations of the CFDs; and, optionally

recording the results as the elements, rᵢⱼ, of a correlation matrix, wherein, e.g., element r₂₆ is the correlation coefficient between the CFD's of the N base pair duplex at position 2 and the N base pair duplex at position 6 of the sequence of interest.

The values of these coefficients determine the similarity or dissimilarity between the N-base nucleic acid sequences present in the sequence of interest. For example, for normalized correlation coefficients, rᵢj=1 indicates that sequences i and j are completely similar, while rᵢⱼ=0 indicates that sequence i and j are completely dissimilar.

These methods can similarly be applied to the comparison of synthetic nucleic acid sequences, e.g., nucleic acid sequences generated according to one of the methods discussed herein, as well as nucleic acid sequences that have been pre-selected, e.g., unique targeting sequences.

In another aspect, the invention features methods of comparing, e.g., analyzing and selecting, nucleic acid sequences. The methods include:

providing a first CFD for a first nucleic acid sequence and a second nucleic acid sequence, wherein the second nucleic acid sequence it the perfect complement of the first nucleic acid sequence;

providing a second CFD for the first nucleic acid sequence and a third nucleic acid sequence, wherein the third nucleic acid differs, e.g., by one or more bases, from the second nucleic acid sequence; and

comparing the first and second CFDs, e.g., in a quantitative manner, e.g., by calculating a correlation coefficient, the variance, or some other statistical measure of similarity.

In preferred embodiments, the CFDs contain data points that are predictions of thermodynamic values, e.g., ΔH, ΔG, ΔS or some combination thereof, corresponding to the different interaction states of the first nucleic acid sequence and the second nucleic acid sequence or the first nucleic acid sequence and the third nucleic sequence. In other embodiments, the CFD contains data points that are predictions of the tₘ associated with the different interaction states of the first nucleic acid sequence and the second nucleic acid sequence or the first nucleic acid sequence and the third nucleic sequence. In a preferred embodiment the calculation of CFD includes accounting for loop structures inferred from mismatches, e.g., more than a selected number of mismatches occurring within a selected distance of one another are considered a loop and matches within that loop are included, calculation of a correlation coefficient for the first and second CFDs. In other preferred embodiments, the comparison involves a calculation of the variance between the first and second CFDs. In still other preferred embodiments, the comparison involves the calculation of more than one measure of similarity. For example, the comparison can include a correlation coefficient, a measure of variance, a value for the amount of nucleotide overlap that occurs upon alignment of the CFDs, e.g., when the CFDs are aligned on absolute minima or maxima for a parameter, the difference in DH of the absolute minima of the CFDs, as well as the number of mismatches that occur in a particular state.

In some embodiments, providing the first and second CFDs involves constructing the CFDs, e.g., as described herein. In other embodiments, the CFDs are stored in a database and are simply retrieved for the purpose of comparison.

In some preferred embodiments, the methods of the invention further include:

constructing a first CFD for a first nucleic acid sequence and a second nucleic acid sequence, wherein the second nucleic acid sequence it the perfect complement of the first nucleic acid sequence;

constructing a second CFD for the first nucleic acid sequence and a third nucleic acid sequence, wherein the third nucleic acid differs, e.g., by one or more bases, from the second nucleic acid sequence;

comparing the first and second CFDs, e.g., in a quantitative manner, e.g., by calculating a correlation coefficient, the variance, or some other statistical measure of similarity; and, optionally

recording the first CFD, the second CFD, a measure of the similarity of the first and second CFDs, or some combination thereof, e.g., in a database.

In preferred embodiments, the quantitative similarity of the shapes of the first and second CFD provides a quantitative indication of the propensity for the first nucleic acid molecule to cross-hybridize with the third nucleic acid molecule. This is useful information when various pairs of strands are simultaneously present in a solution, as is the case in a multiplex environment.

In some preferred embodiments, the methods of the invention can be extended to the analysis of a set of nucleic acid sequences. For example, the methods can be applied to scanning a set of nucleic acid sequences, e.g., taken from a gene, genome, or set of synthetic nucleic acid sequences, and identifying those sequences that have a propensity for cross-hybridizing with nucleic acid sequences complementary to the other sequences in the set. Thus, in some preferred embodiments, the methods of the invention are used to determine the cross-hybridization propensity of a set of nucleic acid sequences, e.g., that are part of a genome, a gene, a selected subset of hybridization probes, or a synthetic set of nucleic acid sequences, using a predefined threshold value(s) for measurements of similarity. The methods include:

providing a set of nucleic acid sequences, e.g., by a method described herein;

providing a CFD for each nucleic acid sequence of the set and a selected group of complements of the nucleic acid sequences of the set (e.g., for the complements of all of the nucleic acids in the population);

comparing the CFD for each nucleic acid sequence of the set and its perfect complement with each of the CFD's for the same nucleic acid and each nucleic sequence of a selected group of complements of the nucleic acid sequences of the set (e.g., for the complements of all of the nucleic acids in the population),

thereby determining the cross-hybridization propensity of a set of nucleic acid sequences.

In preferred embodiments, the comparison can include calculating an M×M matrix, wherein M is the number of nucleic acid sequences in the set. The values in the matrix represent the similarity between the CFD of the nucleic acid sequence, i′, and its perfect complement, i′, and the CFD of the nucleic acid sequence, i, and the complement of a nucleic acid sequence in the set, j′. In related embodiments similarity values are set to 1 or 0, depending upon how they relate to a pre-determined threshold value. For example, correlation coefficients at or above a threshold value of, e.g., 0.6 can be set at 0 (indicating a likelihood of cross-hybridization, and all values below the threshold value can be assigned a value of 1 (indicating a likelihood of non-cross-hybridizing). Threshold values can be adjusted, e.g., based on experimental data or other requirements, e.g., nucleic acid performance requirements, e.g., on a microarray. In preferred embodiments, similarity matrices of this sort are used, e.g., to identify sets of non-cross-hybridizing nucleic acid sequences, as discussed herein.

Any nucleic acid sequence can be analyzed using these methods, including natural nucleic acid sequences of fragments thereof, synthetic nucleic acid sequences, and nucleic acid sequences that have been pre-selected, e.g., unique targeting sequences.

In another aspect, the invention features, a method for analyzing a nucleic acid sequence, to determine the Δ tₘ associated with introducing a change, e.g., a change at a single nucleotide giving rise to a single nucleotide mismatch. The method includes:

providing a nucleic acid sequence A and providing a first CFD for the perfect duplex, AA′;

providing a nucleic acid sequence B′ which is the complement of B and where B differs from A by a change, e.g., a change at a single nucleotide giving rise to a single nucleotide mismatch;

providing a second CFD for the imperfect duplex, AB′;

comparing the first and second CFDs so as to obtain a quantitative measure of their similarity, e.g., a correlation coefficient, or variance, or any other quantitative statistical measure of similarity;

providing a value for a parameter related to stability, preferably tₘ, for the perfect duplex AA′; and

determining a value for the parameter related to stability for the imperfect duplex AB′ using an algebraic expression that includes the parameter related to stability for the perfect duplex AA′ and the measurement of similarity parameter, e.g., a correlation coefficient.

In preferred embodiments, the algebraic expression is linear and includes a correction constant, e.g., as shown in Example 1. In other embodiments, the algebraic expression is non-linear.

Any nucleic acid sequence can be analyzed using these methods, including natural nucleic acid sequences of fragments thereof, synthetic nucleic acid sequences, and nucleic acid sequences that have been pre-selected, e.g., unique targeting sequences.

In another aspect, the methods of the invention are applied to predict the shape of a CFD that corresponds to a desired transition temperature, tₘ, and cross-hybridization propensity. The methods include the following steps:

providing, e.g., preparing, a set of duplex DNA molecules;

providing, e.g., measuring, the melting temperature of each duplex;

determining, e.g., measuring, the cross-hybridization behavior of the set of duplexes;

generating, e.g., calculating, the CFD for each duplex molecules that has been analyzed for melting temperature and cross-hybridization behavior and, e.g., storing them in a database, to provide a training set for an artificial intelligence algorithm;

simplifying the CFD input by finding basis CFD's for the set which are the minimal number of CFD's that can be combined to produce the entire set of CFD's (for example, if three basis CFD's are found then the shape of the CFD for each pair of sequences can be represented by three numbers—the coefficients for the basis CFDs—instead of an entire CFD);

training a neural network or using regression analysis, e.g., multiple regression analysis, to relate the observed transition temperature and cross-hybridization propensity with the coefficients representative of the CFD of each sequence, thereby providing a trained neural network;

optionally, optimizing the trained neural network by interactive adjustment using algorithms, e.g., back propagation and genetic algorithms;

providing values for the desired transition temperature and cross-hybridization propensity to the trained neural network; and

obtaining coefficients for a CFD that is predicted to correspond to the desired transition temperature and cross-hybridization properties; and

using the coefficients and basis CFDs to calculate a predicted CFD for sequences having the desired tₘ and cross-hybridization propensity.

In another aspect, the methods of the invention include predicting the melting temperature, tₘ, and cross-hybridization propensity of nucleic acid sequences from their CFDs. The methods include the following steps:

providing, e.g., synthesizing, a set of duplex DNA molecules;

providing, e.g., determining, the melting temperature of each duplex;

measuring the cross-hybridization behavior of the set of duplexes;

generating, e.g., calculating, the CFD for each duplex molecules that has been analyzed for melting temperature and cross-hybridization behavior and recording, e.g., storing them in a database, the resulting CFDs so as to provide a training set for an artificial intelligence algorithm;

simplifying the CFD input by finding basis CFD's for the set which are the minimal number of CFD's that can be combined to produce the entire set of CFD's (for example, if three basis CFD's are found then the shape of the CFD for each pair of sequences can be represented by three numbers—the coefficients for the basis CFDs—instead of an entire CFD);

training a neural network or using regression analysis, e.g., multiple regression analysis, to relate the coefficients of each sequence with the observed transition temperature and cross-hybridization propensity, to thereby provide a trained neural network;

optionally, optimizing the trained neural network by interactive adjustment using algorithms (e.g.,. back propagation, genetic algorithms etc.); and

predicting the transition temperature and cross hybridization propensity for any new sequence from the coefficients of the basis CFD's for that sequence.

In another aspect, the methods of the invention can be used to scan a nucleic acid sequence of interest, e.g., a gene or genome sequence, for optimal regions for micro-array applications. Often desired optimal characteristics for microarray applications are that the sequences used be isothermal (i.e., the tₘ of all probes on the array need to lie in a narrow temperature interval) and that they have low cross-hybridization propensity. Methods of the invention that help achieve this include the following steps:

defining the tₘ at which the micro array will be operated;

defining the desired threshold for cross-hybridization propensity;

defining the length of the probes for the microarray;

using a trained neural network, e.g., made as described herein, to predict the coefficients of the basis CFD's from the desired tₘ and cross-hybridization propensity;

using the basis CFD's and coefficients to generate a predicted CFD matching the desired tₘ and cross-hybridization propensity;

examining all sequences (e.g, in a set of genes or in a genome) of the desired length and providing their CFD's;

determining the similarity between provided CFDs and the predicted CFD;

labeling each position (e.g., of the genome) by its corresponding correlation coefficient;

defining a threshold of similarity, e.g., a correlation coefficient rᵢⱼ>0.7,

thereby providing sections of the gene above this threshold and having the desired tₘ and cross-hybridization propensity.

In another aspect, the methods of the present invention are useful for generating synthetic nucleic acid sequences. Nucleic acid sequences of length N (e.g., N=2 to 200) are built from a set of possible nucleotide base monomer units, e.g., A, G, C, T, and/or any other base monomer, to have predefined composition and properties. Thus, in some embodiments, the methods include:

specifying the sequence length N and, optionally, the desired % G−C;

determining one or more base compositions, e.g., numbers of A, T, C, and G bases, of the synthetic nucleic acid sequences that satisfy the sequence length condition and, if applicable, the % G−C condition;

providing, for each base composition, a partial representation, e.g., a partial mathematical representation, e.g., an incomplete sequence graph or n×n matrix (where n is the number of different types of bases in the nucleic acid sequence, e.g., n=4 for DNA), corresponding to a set of synthetic nucleic acid sequences that have the same base composition;

partitioning, for each base composition (or partial representation), the bases, e.g., A, T, G and C, into many different, e.g., all possible, nearest neighbor connections that satisfy the sequence length and base composition conditions, thereby providing for each partial representation a set of complete representations, each of which corresponds to an isothermal (within the limits of the nearest-neighbor approximations) set of nucleic acid sequences; and

enumerating all of the isothermal nucleic acid sequences defined by each complete representation, thereby generating a set of synthetic nucleic acid sequences.

In preferred embodiments, the nucleic acid sequence length N is about 15 to 200 bases, more preferably about 20 to 100 bases, and most preferably about 25 to 75 bases.

In preferred embodiments, the GC content (% G−C) of the nucleic acid sequences is 50% +/−20%, 10%, or 5%. In other preferred embodiments, the G and C content of the nucleic acid sequences is each 25% +/−10%, or 5%. In still other preferred embodiments, the A, T, G, and C content of the nucleic acid sequences is each 25% +/−10%, or 5%.

In preferred embodiments, all of the possible base compositions that satisfy the sequence length and base composition conditions, e.g., % G−C, G and C composition, or A, T, G, and C composition, are determined.

In preferred embodiments, the representation of base composition is a M×M matrix (wherein M corresponds to the number of different bases that are included in the nucleic acid sequences) or a sequence graph that is Eulerian. In particularly preferred embodiments, the representation of base composition is a 4×4 Eulerian matrix, e.g., as described herein. In some embodiments, the rows and columns in the matrix are defined, e.g., the matrix can be labeled ATGC×ATGC, wherein the 1,1 position gives the number of A's. In other embodiments, the rows and columns are arbitrary, e.g., ijkl×ijkl, and the identity of the bases is not assigned until after sequences (in the ijkl format) have been extracted from the matrices.

In preferred embodiments, the partitioning of the bases with respect to nearest-neighbor connections is performed in all possible ways such that all possible distributions of nearest-neighbor connections are sampled.

In preferred embodiments, the complete nucleic acid sequence representations are enumerated, in part, by determining the basic sequence cycle compositions of the sequence representations, e.g., as described herein.

In other embodiments, instead of starting from an adjacency matrix, the process of generating nucleic acid sequences starts from a cycle coefficient vector.

These methods can be used to supply nucleic acid sequence for use in other methods described herein.

In another aspect, the methods of the invention are useful for providing a population of synthetic nucleic acid sequences and include: providing a value N for the length of a nucleic acid sequences;

providing values for the base composition of the nucleic acid sequences, e.g., the base composition can be 25% +/−5% for each of the four bases, provided that the total is 100%;

providing a representation, sometimes referred to herein as a Eulerian representation, of possible sequences which representation can be described by a Eulerian graph; and

extracting sequences from the representation,

thereby providing a population of synthetic nucleic acid sequences.

In preferred embodiments, the nucleic acid sequence length N is about 15 to 200 bases, more preferably about 20 to 100 bases, and most preferably about 25 to 75 bases.

In preferred embodiment the representation can be a matrix, e.g., an M×M matrix, wherein M is equal to the number of bases used, and wherein each number in the diagonal is the number of the corresponding residues in each member of the set of isothermal sequences. In preferred embodiments, M=4 and, e.g., the four bases are A, T, G, and C. In some embodiments, the rows and columns in the matrix are defined, e.g., the matrix can be labeled ATGC×ATGC, wherein the 1,1 position gives the number of A's. In other embodiments, the rows and columns are arbitrary, e.g., ijkl×ijkl, and the identity of the bases is not assigned until after sequences (in the ijkl format) have been extracted from the matrices.

In preferred embodiments, the methods of the invention optinally include limiting the number of allowed nucleotide repeats, e.g., AA, CC, TT, or GG sequence elements.

In a preferred embodiment extracting the sequence can include decomposing the Eulerian representation into components, e.g., component matrices corresponding to the basic cycle, and permuting the components to produce the population of sequences.

These methods can be used to supply nucleic acid sequence for use in other methods described herein.

In another aspect, the methods of the invention are useful for providing a population of synthetic nucleic acid sequences and include:

a) providing a value N for the length of the nucleic acid sequences;

b) providing values for the base composition, e.g., a base composition of about 25+/−5% for each of the four bases, provided that the total is 100%;

c) providing a representation, sometimes referred to herein as a Eulerian representation, of possible sequences which representation can be described by Eulerian graph;

d) repeating steps a, b, and c, at least one time, and preferably a sufficient number of times to provide at least 1000 matrices; and

e) extracting sequences from the representations, thereby providing a population of synthetic nucleic acid sequences.

In preferred embodiments, the nucleic acid sequence length N is about 15 to 200 bases, more preferably about 20 to 100 bases, and most preferably about 25 to 75 bases.

In preferred embodiments, the value for the length of the nucleic acid is the same in each of the different Eulerian representations, but in other embodiments it can differ.

In preferred embodiments, the value for the base composition is the same in each of the different Eulerian representations, but in other embodiments it can differ.

In preferred embodiment the representation can be a matrix, e.g., an M×M matrix, wherein M is equal to the number of bases used, and wherein each number in the diagonal is the number of the corresponding residues in each member of the set of isothermal sequences. In preferred embodiments, M=4 and, e.g., the four bases are A, T, G, and C. In some embodiments, the rows and columns in the matrix are defined, e.g., the matrix can be labeled ATGC×ATGC, wherein the 1,1 position gives the number of A's. In other embodiments, the rows and columns are arbitrary, e.g., ijkl×ijkl, and the identity of the bases is not assigned until after sequences (in the ijkl format) have been extracted from the matrices.

In preferred embodiments, the methods of the invention optinally include limiting the number of allowed nucleotide repeats, e.g., AA, CC, TT, or GG sequence elements.

These methods can be used to supply nucleic acid sequence for use in other methods described herein.

In another aspect, the methods of the invention are used to generate probe sequences for use, e.g., in a universal sequence microarray. The methods include:

generating an Eulerain representation, e.g., an Eulerian graph, describing a plurality of nucleic acid sequences;

partitioning the nucleic acid sequences according to a given base composition, e.g., roughly equal base representation, e.g., 25% +/−10% for each base (assuming that there are four bases);

creating subgraphs that specify how many and what type of monomeric bases comprise the sequences, wherein the subgraphs have vertices that correspond to the types of oligomeric sequences and edges that correspond to partitioning of the integers that describe properties of the sequences;

characterizing the sequences by their propensity for cross-hybridization by (i) formulating the context functional descriptor of each sequence aligned with its perfect complement as a nucleic acid duplex at each alignment position, and (ii) assigning a number representing the relative thermodynamic stability of the duplex, thereby generating diagonal elements of a correlation matrix; and

(e) aligning the deepest minima of off-diagonal elements of the correlation matrix with the deepest minima of the diagonal elements of the correlation matrix, thereby analyzing the potential interactions between the nucleic acid sequences.

In a preferred embodiment the method analyzes the potential interactions between nucleic acid sequences, e.g., sequences described herein, wherein the subgraphs generated in step (c) are listed in a relative manner according a desired property, e.g., isothermal character or potential for cross-hybridization.

In another aspect, the invention features, a method for analyzing nucleic acid sequences, e.g., analyzing the potential interactions between nucleic acid sequences. The method includes the steps of:

(a) generating an Eulerian graph, or representation thereof, describing a plurality of nucleic acid sequences;

(b) optionally, partitioning the nucleic acid sequences according to a given composition;

(c) creating subgraphs that specify how many and what type of the monomeric basis comprise the sequences wherein the subgraphs have vertices that correspond to the types of oligomeric sequences and edges that correspond to partitioning of the integers that describe properties of the sequences;

(d) characterizing the sequences by their propensity for cross-hybridization by (i) formulating the context functional descriptor of each sequence aligned with itself as a nucleic acid duplex at each alignment position, and (ii) assigning a number representing the relative thermodynamic stability of the duplex, thereby generating diagonal elements of a correlation matrix;

(e) characterizing the sequences by their propensity for hybridization by (i) formulating the context functional descriptor of each sequence aligned with every other sequence as a nucleic acid duplex at each alignment position, and (ii) assigning a number representing the relative thermodynamic stability of the duplex, thereby generating off-diagonal elements of the correlation matrix; and

(f) aligning the deepest minima of off-diagonal elements of the correlation matrix with the deepest minima of the diagonal elements of the correlation matrix, thereby analyzing the potential interactions between the nucleic acid sequences.

In another aspect, the invention features a method of and identifying a population of sequences, e.g., to provide a subpopulation that has a selected property, e.g., a tₘ within a pre-selected range. The method includes:

providing an initial population of nucleic acid sequences, e.g., cDNAs;

providing, for a first nucleic acid sequence of the population, a selected set of oligomers derived from the first nucleic acid, e.g., providing all or a subset of all possible oligomers of a preselected length, e.g., all possible oligimers of suitable length for use as a capture probe on an ordered array on nucleic acids, e.g., a microarray described herein, or useful for amplification reactions, e.g., PCR;

providing, for a second and optionally subsequent nucleic acid sequence of the population, a selected set of oligomers derived from the second or subsequent nucleic acid, e.g., providing all or a subset of all possible oligomers of a preselected length, e.g., all possible oligomers of suitable length for use as a capture probe on an ordered array on nucleic acids, e.g., a microarray described herein, or useful for amplification reactions;

providing a tₘ, preferably by calculation, using e.g., art-known methods, for oligos produced above and its perfect compliment;

sorting the oligomers for which a tₘ is provided into a plurality of subpopulations each having a preselected range of values for tₘ. The method can include sorting the oligomers into groups or bins having a preselected range on values for tₘ, and optionally, finding a target population by moving a window of a preselected temperature range along the groups or bins,

thus providing a subpopulation which has a selected property, e.g., a tₘ within a preselected range. This method can be used to provide a population of nucleic acids for use in other methods described herein.

In preferred embodiments, providing means providing in a computation form, e.g., in silico, as opposed to providing actual molecules of the substance.

In another aspect, the invention features a method of representing a set of nucleic acid sequences, e.g., for use in computational algorithms, wherein the representation is a M×M matrix that corresponds to a Eulerian sequence graph, and wherein M is equal to the number of bases present in the nucleic acid sequences, e.g., four in the case of DNA.

In another aspect, the invention features a method of representing a set of nucleic acid sequences, e.g., for use in computational algorithms, wherein the representation is a twenty-four element cycle coefficient vector, and wherein each element of the vector corresponds to a particular basic sequence cycle, e.g., as defined herein.

In another aspect, the invention features, a file, e.g., a computer readable file, having a record which includes an element which identifies a nucleic acid and an element which describes the CFD, or one or more components thereof. In a preferred embodiment, the record includes an element which identifies a property of the nucleic acid or the peptide it encodes, e.g., the ability of the nucleic acid to interact with another molecule, e.g., a protein (e.g., a transcription factor, a histone, or a ribosomal protein), another nucleic acid molecule, or a chemical compound. In preferred embodiments the file includes records for a plurality of nucleic acids. The file can have records from any of the populations of nucleic acid described herein.

In another aspect, the invention features a set of nucleic acids, generated or compiled by a method described herein, e.g., useful as a set of probes or an ordered array, e.g., on a microarray.

Methods of the invention rely on a parameter termed Context Functional Descriptor, As discussed in more detail herein, CFD-based analysis allows for consideration of the complete oligomeric “context” or “all neighbors” influence of a nucleic acid sequence, as opposed to merely relying on nearest-neighbor and next-nearest neighbors interactions, as done in many of the prior art methods. As is shown below, it can be used in a variety of methods, including: methods for determining the stability of nucleic acid duplexes and parsing nucleic acids into isostable groups; methods for analyzing the likelihood of cross-hybridization in mixed samples. The invention also provide methods which use Eulerian constructs to generate or analyze nucleic acid sequences.

Methods of the invention provide for the generation of databases of oligomeric nucleic acid sequences that have a number of useful properties and can thus be used for various applications, e.g., on DNA micro-arrays related, e.g., to diagnostic applications, assays, drug discovery, and genetic screening. Sets of nucleic acid sequences are useful in many applications, e.g., in micro-arrays or other multiplex tools or methods where many nucleic acid molecules hybridize in parallel. Methods of the invention allow for the provision of sets of nucleic acid oligomers which meet one or more of the following conditions:

1. The stability of all duplexes in the set is within preselected values, preferably all are very similar, and more preferably all are essentially identical. (Micro-array hybridization reactions are generally performed at constant temperature T_{work} for all DNA, so one does not want a subset of lower stability being, e.g., 30% melted, and another higher-stability subset being 100% hybridized at T_{work}.

2. Members of the set are selected to minimize cross-hybridization such that there is less than a preselected amount of cross-hybridization. Ideally, the complementary strands of members of the set are capable of hybridizing only with their perfect complement (target) and not with any of the other members of the set (zero cross-hybridization condition). This helps ensure that there are no errors, e.g., false positives, in the applications.

3. Oligomers in the set are informationally highly relevant. They are relevant and preferably unique, or represent unique and diagnostic genes or parts thereof, e.g., mutational hot spots or SNPs, which are essential for assays, recognition, and forensic applications.

One can start with a raw set of oligomers (i.e., a set which does not necessarily obey conditions 1-3) and select a subset of them having one or more, and preferably all, of these properties. This is accomplished by the application of a functional representation of nucleic acid sequence, referred to herein as a Context Functional Descriptor, or CFD. Each oligomer i from the raw set has its own characteristic CFD_{ii′} which can be calculated by using its ideal complement i′. The Minimum of CFD_{ii′} (or maximum, if CFD_{ii′} is expressed in melting temperature units) represents the stability of each DNA duplex in the set and can be thus used to parse the raw set into desired subsets of iso-stable oligomers, if necessary. Differences of shapes of CFD_{ii′} and CFD_{jj′} (where i is different from j) can be used to quantify more subtle stability differences between oligomeric duplexes ii′ and jj′.

Embodiments of the invention incorporate a consideration of the stabilizing role of mismatched areas called loops into the CFD. Consider, for example,

˜CACC˜
˜GCTG˜

The A, T arrangement in the above sequence forms a stabilizing “loop” where conventional methods consider two (destabilizing) mismatches.

To select for sets which fulfill condition 2, methods of the invention use a cross-hybridization set of CFD_{ij′} for each oligomer i. These CFD's are determined using oligomer i and complements of all other oligomers j′ in the set (where i is different from j). The shape of CFD_{ii′} can be interpreted as a representation of the ideal hybridization energy landscape for oligomer I, and can be used as a reference.

The possibility that oligomer i will cross-hybridize with wrong complement j′ was found to be proportional to the similarity of shape of cross-hybridization CFD_{ij′} to the (reference) shape of perfect duplex CFD_{ii′}. Methods disclosed herein define quantitative similarity measures for CFD_{ii′} and CFD_{ij′} These measures allow for the selection of non-cross-hybridizing nucleic acid sequence subsets from the iso-stable raw subset by application of relevant threshold conditions. The cross-hybridization propensities obtained from comparisons of CFD_{ii′} and CFD_{ij′} are pair-wise properties for different pairs of oligomers i and j.

Condition 2 requires that a relationship that is valid for a pair of oligomers, ij, from the set, e.g., they are non-cross-hybridizing, is also valid for all other pairs involving oligomers i and j. Thus, it is necessary to convert pair-wise relationships between oligomers into the collective property of the ideal set.

Methods described herein provide a novel approach to this problem, referred to herein as ‘crystallization’. This effective method utilizes in the first step the clique algorithm that selects a non-cross-hybridizing ‘core’ subset of oligomers from the processed raw set. In the second step, the remaining oligomers from the raw set are compared via their CFD_{ij′} to all members of the core and are added to the core only if the similarity between the reference CFD and CFD_{ij′} fulfills a threshold criteria (e.g., the remaining oligomers are added to the core providing that they are non-cross-hybridizing with all members of the core).

This two-step process allows these calculations to be performed in a reasonable amount of time; clique algorithm processing time increases nonlinearly with the number of processed sequences, so the small constant core size allows one to keep that time constant for all oligomer sizes. ‘Crystallization’ processing time is linear or sub-linear in the number of processed sequences, as increases in core size during the process is compensated by the fact that the processing of any given sequence ends at the first un-acceptable comparison.

To treat condition 3, it is important to recognize that there are two types of nucleic acid oligomers. In the first category are nucleic acid oligomers that are not obtained from natural sequences (e.g., form genes or chromosomes). In the second category are nucleic acid oligomers that are part of some natural sequence, e.g., a genome.

Condition 3 is already fulfilled, for sequences that are not obtained from natural sequences, by selection of a non-cross-hybridizing subset of the raw set. The informational uniqueness of a linear polymer of four monomers (bases) originates in the fact that it has minimal sequence homology to any other polymer in the set. Non-cross-hybridization condition maximizes number of sequence differences in the ideal set and thus ensures that each member is also informationally unique.

To fulfill condition 3 for sequences of natural origin, methods described herein make use of weighting profiles that are overlaid over the parent natural DNA sequence and that have maxima at the informationally important and/or unique sequence positions and the selection of the oligomers into the raw set is modified to reflect these maxima. These weighting profiles can be determined experimentally, can emphasize protein-coding region, or can relate to the context of the sequence itself.

Thus, methods of the invention also relate to the optimal selection of the raw set of nucleic acid sequences, e.g., oligomers. Methods of the invention avoid drawbacks of other methods of generating sequences, e.g., obtaining the raw set by systematic generation of all possible permutations of the four bases in the oligomer positions. This approach is not efficient because of the enormous combinatorial complexity that results in practically un-treatable sizes of the raw set even for moderate oligomer lengths. At the same time, most of these systematically generated sequences will violate conditions 1-3 and would be thus rejected in the selection anyway.

Methods of the invention provide targeted generation of nucleic acid sequences that obey one or more of conditions 1 to 3. The methods are different for the two types of DNA oligomers (synthetic and natural, as defined above).

For DNA oligomers from the first category, the method uses the fact that a mathematical object, e.g., a Eulerian graph with four vertices, can represent any nucleic acid sequence, e.g., DNA. These methods rely on the realization that once the Eulerian graph is created for any nucleic acid sequence, that graph represents not only that particular DNA oligomer, but also many other oligomers of the same length that are iso-stable up to the nearest neighbor contextual level.

One can generate a set of all sequences that fulfill condition 1 by extracting them from single Eulerian graph. Methods of the invention provide for the extraction of these sequences, e.g., with optimally working algorithms. Each sequence is a path in the given Eulerian graph. The multiplicity of possible paths in the graph gives the multiplicity of the sequences that can be extracted from it. Methods of the invention first decompose the graph or equivalent Eulerian representation, e.g., a matrix, into cyclic sub-paths—linear combinations of up to 24 base cycles—that are then efficiently encoded in the numerical structure and combined according to specified rules. This reduces the number of steps required in existing methods of finding paths in Eulerian graphs.

Methods of the invention allow for the generation of improved DNA oligomer databases for all practically feasible sequence lengths by generating all Eulerian graphs for a given sequence length. This method utilizes the fact that an M×M integer matrix can uniquely and unequivocally represent a Eulerian graph having M indices. Elements of the matrix are related by a series of conditions that reflect the unique molecular structure of the linear polymer. To eliminate matrices that would generate mostly sequences that violate condition 2 (non cross-hybridization) the invention uses additional conditions that supplement those mentioned above. Thus, methods of the invention provide for the generation of a raw set for DNA oligomers from the first category.

To generate the raw set of DNA oligomers from the second category, (natural sequences) the methods of the invention use both the non-weighted and profile-weighted approaches for the initial step of the process. For the non-weighted case, the sequence is decomposed systematically into oligomers of a specified length, moving k bases at a time, where k can be one or larger. For the profile-weighted case, the decomposition of the natural DNA sequence into oligomers of length N starts at position Pᵢ-N, where Pᵢ is the position for which the corresponding profile has one of its maxima and the sequence is decomposed systematically k bases at a time until Pᵢ is reached. Algorithm then moves to position of the next maximum, Pᵢ₊₁ and the process is repeated. (This ensures that the ‘critical’ sequence positions characterized by the profile maximum are present in all raw set oligomers).

Subsequent steps can be identical for both categories. Raw oligomeric sequences are sorted into iso-stable groups using their CFD_{ii′}. Cross-hybridization CFD_{ij′}are then calculated within each iso-stable subgroup, the results are used to create a ‘core’ subset, and by crystallization the final ideal set of oligomers is determined. To maximize the size of the ideal set, the process starts with the analysis of the frequency of occurrence of different stabilities of DNA oligomers along the sequence. Oligomers with the most frequent stability are then processed in the above-described way.

Using graphs to encode the iso-stable (iso-thermal) nucleic acid sequences is a valuable tool for use in genomics—the fact that an enormous numbers of sequences (up to 10²⁰ in some cases) can be represented using a single computer-encoded entity (16 numbers for an adjacency matrix or 24 numbers for a cycle coefficient vector) and that this representation contains information about details of the stability of the sequences, makes this representation attractive for computational methods, including genomics applications and data mining.

Thus, the invention features a method of analyzing a nucleic acid sequence, including: providing a sequence graph representation, e.g., a Eulerian representation of a population of sequences, wherein the population includes at least 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰ sequences; and searching the population for a sequence of interest or comparing a reference sequence with a sequence in the population.

The existence of this condensed representation of each entry in databases of nucleic acid sequences enables one to: a) find novel relationships between natural and synthetic nucleic acid sets; and b) generate ‘naturally-biased’ synthetic nucleic acid sequence sets (after the natural sequence is processed, the cycle coefficient vector of each stored natural oligomeric sequence is inserted into the universal SEQ-TG™ algorithm and all ideal oligomeric sequences are generated from it).

Due the quantitative treatment of sequence context, the present invention has many benefits and advantages, several of which are listed below.

A benefit of the invention, as related to nucleic acid sequence analysis (e.g., predicting nucleic acid hybridization), selection (e.g., based on tₘ and non-cross hybridizing behavior), and generation, is that the present invention obviates the need to consider or evaluate, explicitly, order dependent sequence specific interactions (e.g., singlet, nearest-neighbors, and next-nearest-neighbors interactions). Instead, existing quantitative parameters that consider or describe order dependent interactions serve as the starting point for constructing the CFD, and are thus intrinsic components of the methods of the invention.

Another advantage of the present invention, as related to nucleic acid sequence analysis, selection, and generation, is that the methods have enhanced predictive power over existing analytical methods. Known parameters for analyzing nucleic acid sequences (e.g., nucleic acid hybridization), and the information embodied in them, are included in the present methods and, in addition, the overall influence of sequence context is considered.

A further benefit of the methods of the present invention, as related to nucleic acid sequence analysis, selection, and generation, is that they provide a more precise means by which to characterize sequences and find sequences with similar sequence dependent properties.

A still further benefit of the present invention is that the methods for sequence analysis, selection and generation serve the needs of today's large-scale, high-throughput commercial applications of nucleic acid hybridization.

Another advantage of the present invention is that it permits the design of microarrays with superior hybridization characteristics.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts the differential melting curves for the three pairs of nucleic acid oligomers, PM (perfect match), MM (L) (mis-match left), and MM (R) (mis-match right), as discussed in Example 1.

FIG. 2 depicts various alignment positions between two nucleic acid sequences, each 31 bases in length, as shown in Example 1. The numbers along the right-hand side show the alignment position of the bottom strand relative to the position along the top strand, moving from position 1 to position 2 to position 31 to position 35 to position 61.

FIG. 3 depicts the CFD constructed for each duplex sequence of Example 1: PM, MM (L), and MM(R). The corresponding CFD's are expressed in terms of the calculated tₘ of each alignment point.

FIG. 4 depicts the actual melting curves for various hybrid pairs, as discussed in Example 2. The top portion of FIG. 4 illustrates the overlay of four melting curves: the solid line is I_{T} with I_{P}; the dashed line is II_{T} with II_{P}; the dotted line is I_{T} with II_{P}; and the dotted/dashed line is II_{T} with I_{P}. The lower portion of FIG. 4 also illustrates the overlay of four melting curves: the solid line is IV_{P} with IV_{T}; the dashed line is III_{P} with III_{T}; the dotted/dashed line is IV_{T} with III_{P}; and the dotted line is III_{T} with IV_{P}.

FIG. 5 depicts the context functional descriptors plotted as relative thermal stability (designated tₘ in degrees centigrade) of the model nucleic acid hybrid duplexes discussed in Example 2 at the various alignment positions for the following nucleic acid pairs: at the top is I_{T} with I_{P}; second from the top is II_{T} with II_{P}; second from the bottom is III_{T} with III_{P}; and at the bottom is IV_{T} with IV_{P}. These duplexes are designed to have only conventional (GC or AT) base pairing and no mismatches.

FIG. 6 depicts the context functional descriptor (CFD) of the nucleic acid duplexes discussed in Example 2. These duplexes have mismatches. The solid lines show the CFD for the duplexes, which takes into consideration the number of complementary base pairs, nearest neighbor stacking interactions, and base pair mismatches present at each alignment position. The dotted lines show the CFD for the duplexes with an additional thermodynamic contribution of additional stabilizing interactions. The predicted melting temperatures calculated using CFDs that account for additional stabilizing interactions more closely approximated the observed duplex melting temperatures.

FIG. 7 depicts potential stabilizing interactions in the mismatch duplexes discussed in Example 2.

FIG. 8 depicts a flow chart of the method of the invention embodied in the SEQ-TG™ process described herein. The calculations and analysis operations are carried out on a computer.

FIG. 9 depicts the twenty-four basic cycles representing DNA sequence, and their corresponding adjacency matrices.

FIG. 10 depicts a Eulerian sequence graph corresponding to a set of DNA sequences (lower right hand corner) and its decomposition into a set of basic sequence cycles.

FIG. 11 depicts a cycle coefficient vector and its relationship to the set of basic sequence cycles that are part of the corresponding Eulerian sequence graph.

FIGS. 12A, B, and C depict how the basic cycles of DNA sequence are used to decompose a sequence graph and the linking of the basic cycles (or permutable sequence units) to enumerate the different nucleic acid sequences represented by the sequence graph. Further explanation of FIG. 12 is presented in the text.

FIG. 13 depicts a flowchart representing the steps in the SEQ-TGTM algorithm used to generate synthetic nucleic acid sequences.

FIG. 14 depicts the fourteen linearly independent basic cycles representing DNA sequence. The other ten basic cycles shown in FIG. 9 are linear combinations of these basic cycles.

Like reference symbols in the various drawings indicate like elements.

### DETAILED DESCRIPTION

The present invention provides methods and means for generating, analyzing and selecting nucleic acid sequences. In preferred embodiments the methods are useful for the analysis and selection of natural nucleic acid sequences, e.g., sequences occurring in nature, e.g., sequences present in genomic DNA, cDNAs, ribosomal RNAs, mRNAs, SNPs, or mutational hot spots. In other preferred embodiments, the methods are useful for the generation, analysis and selection of synthetic nucleic acid sequences, e.g., sequences generated computationally or sequences that include non−naturally occurring bases (e.g., inosine) or peptide nucleic acids (PNAs). The selected nucleic acid sequences can, for example, be used in hybridization-based nucleic acid technologies, such as microarray analysis or the amplification of nucleic acid molecules.

The methods of the invention allow for the precise design and/or selection of sequences for use in an assay to amplify or capture a single preferred, or suite of preferred, “target” sequences that the assay seeks to detect or quantify.

In particular, the present invention is useful for designing and/or selecting sequence(s) with the highest hybridization fidelity, and is enabled by heightened quantitative insights regarding the influence of sequence context on sequence dependent hybridization behavior of nucleic acid sequences. All nucleic acid hybridization reactions are sequence dependent. Therefore, understanding the effects of sequence context in hybridization is critical to the design of optimally reliable, efficient and economical microarray-based assays. The present invention is different from microarray hardware and software engineering systems (in situ synthesis, various spotting technologies or image analysis, etc.), as it concerns the identification of sequences that exhibit high fidelity hybridization.

The methods and tools of the present invention are built upon the premise that a potentially very important but little appreciated or understood component of nucleic acid sequence dependent stability and hybridization is sequence context. The nucleic acid analysis methods and tools of the present invention are used to analyze sequence context and associated thermodynamic properties. In some embodiments, the present invention provides a robust quantitative sequence design tool for the generation and selection of optimum sequences for use in highly parallel multiplex hybridization reactions.

The methods and tools of the present invention also provide a means to evaluate the context dependent contributions to thermodynamic stability of duplexes having single base pair mismatches and evaluate correction factors, that account for context effects, necessary to augment conventional calculations for more accurate predictions of sequence-dependent stability. Solution melting experiments of some specifically designed duplexes are discussed in Example 1. The data from such specifically designed duplexes demonstrate applications of the method.

In some embodiments, the methods and tools of the present invention contemplate the selection of optimum sets of sequences from known target sequences, e.g., by evaluating the cross-hybridization potential of the known target sequences using CFDs. In other embodiments, the methods and tools of the present invention can be used to generated a non-random set of nucleic acid sequences, e.g., using the methods described below, and then select optimal subsets of sequences from the set, e.g., by evaluating the cross-hybridization potential of the sequences of the set using CFDs. The methods and tools of the present invention also permit adjustments for observed experimental results based on the hybridization properties of a sample of the selected sequences.

The present invention can be applied to DNA/DNA hybrids, RNA/RNA hybrids, DNA/RNA hybrids, hybrids involving nucleic acid base analogs or peptide nucleic acids (PNA's), and any other type of nucleic acid hybrid.

As used herein, the term “hybridization” refers to the pairing of complementary nucleic acid sequences, e.g., perfect complements, as well as non-complementary nucleic acid sequences, e.g., sequences that, when bound to one another, contain one or more base-pair mismatches. Hybridization and “strength of hybridization” (i.e., the strength of the association between two nucleic acid sequences, commonly characterized by the Tₘ or melting temperature) is impacted by many factors well known in the art. These include the degree of complementarity between the nucleic acid sequences, the % G−C content and associated thermodynamic stability, and the stringency of conditions that can be affected by experimental conditions. Conditions that impact stringency include, e.g., the concentration of each nucleic acid sequence, solvent ionic strength (e.g., salt concentration), and the presence of co-solutes (e.g., the presence or absence of osmolytes such as polyethylene glycol, binding ligands such as distamycin, ethidium, single strand binding proteins, and restriction enzymes).

As used herein, “sequence context” refers to the collective properties associated with a linear nucleic acid polymer sequence, including the bases present in the sequence (e.g., Adenosine (A), Thymine (T), Cytosine (C), Guanosine (G), Uracil (U), and deoxy forms thereof, as well as non−natural bases or base analogs such as Inosine (I), etc.), the base composition (e.g., % A, % T, % C, and % G in the case of DNA or RNA), and the relative position of the bases with respect to one another.

As used herein, a “context functional descriptor” or “CFD” consists of two or more points of data that provide an estimate of the strength of hybridization for a pair of nucleic acid sequences, wherein estimates of the strength of hybridization for at least two distinct interaction states of the pair of nucleic acid sequences are included in the CFD. When the term CFD is used in reference to a single sequence, it should be understood that the CFD consists of two or more points of data that provide estimates for the strength of hybridization of the nucleic acid sequence and its perfect complement. A CFD can be generated for any pair of DNA sequences. Comparison and/or selection of sequences is based upon the properties of the their respective CFD's. In a preferred embodiment, the estimates of the strengths of hybridization are thermodynamic estimates, e.g., ΔG, ΔH, ΔS, equilibrium constant, and/or tₘ. In preferred embodiments, a CFD will include at least N+M−1 data points, wherein N and M are the respective lengths of the nucleic acid sequences that make up the pair of nucleic acid sequences.

As used herein, a “perfect complement” is a nucleic acid sequence that is the same length as a first nucleic acid sequence and which can bind to the first nucleic acid sequence without any base-pair mismatches occurring.

Thermodynamic Stability of Nucleic Acid Duplex Molecules

The hybridization of two nucleic acid sequences to form a duplex molecule involves sequence-dependent interactions between the two nucleic acid sequences of the duplex. Sequence-dependent stability of duplex DNA has been a topic of theoretical and experimental investigation for nearly fifty years. Wartell, R. M. and Benight, A. S. (1985) “Thermal Denaturation of DNA Molecules: A Comparison of Theory with Experiment”. Physics Reports (126) p. 67-107. Over that period a variety of different models and analytical procedures have been applied to evaluate sequence-dependent thermodynamic stability parameters. Those studies have been inspired by the hope of being able to predict correctly the outcome of melting experiments (thermodynamic stability) from sequence alone. This hope has yet to be fulfilled entirely. Over the course of time, DNA samples studied for evaluation of stability parameters have varied from long viral or bacterial genomes, to shorter restriction fragments, synthetic repeating sequence polymers, short oligomers and dumbbells. Doktycz, M. J., et al. “Studies of DNA Dumbbells I: Melting Curves of Sixteen DNA Dumbbells With the Sixteen Base-Pair Duplex Stem Sequence 5′-GTATCCXYXYGGATAC-3′ (X, Y=A, T, G, C) and T4 End-Loops: Evaluation of the Nearest-Neighbor Stacking Interactions in DNA”. Biopolymers, 32:849-864 (1992); Owczarzy, R., et al. “Predicting Sequence Dependent Melting Stability of Short Duplex DNA Oligomers” Biopolymers 44:217-239 (1997); Benight, A. S., et al. “Sequence Context and DNA Reactivity: Application to Sequence-Specific Cleavage of DNA”. Adv. Biophys. Chem., 5:1-55 (1995).

Initially, the sequence dependence of DNA stability was found, to a first order approximation, to be a linear function of the relative fractions of A−T and G−C type base pairs. With improved experimental resolution in the art, higher sample quality and well-designed sequences, the evaluation of higher order (i.e. nearest neighbor) sequence-dependent interactions in duplex DNA with statistical accuracy, was enabled. To date, at least eleven different sets of nearest-neighbor, sequence-dependent interactions have been reported. Some of these have been compared. Benight, A. S., et al., Adv. Biophys. Chem. (5) p. 1-55 (1995); SantaLucia J. Jr., Proc. Nat'l Acad. Sci., U S A., 95:1460-1465 (1998). The most notable parameters, evaluated from melting studies of short linear duplex DNA oligomers, are the nearest-neighbor stacking and mismatch parameters reported by SantaLucia and coworkers. SantaLucia J. Jr., “A unified view of polymer, dumbbell, and oligonucleotide DNA nearest-neighbor thermodynamics,” Proc. Nat'l Acad. Sci., U S A., 95:1460-1465 (1998). The SantaLucia parameters are used in the predictive algorithm HyTher™ to calculate the thermodynamic stability of short duplex DNA oligomers. Both perfect matched duplexes and duplexes having single base mismatches can be considered using HyTher™. Predictions from HyTher™ for standard duplexes are, in most cases, fairly accurate. However, as has been pointed out, using either the SantaLucia parameters or other stability parameters derived from melting analysis of DNA dumbbells or other sets of nearest-neighbor parameters from other laboratories, many exceptions can be readily found where accurate tₘ's cannot be predicted. Owczarzy, R., et al. “Predicting Sequence Dependent Melting Stability of Short Duplex DNA Oligomers,” Biopolymers 44:217-239 (1997); Owczarzy, R., et al. “Studies of DNA Dumbbells VII: Evaluation of the Next-Nearest Neighbor Sequence Dependent Interactions in Duplex DNA,” Biopolymers (Nucleic Acid Sciences), 52:29-56 (2000). The precise physical/chemical origins of these exceptions could be due to a number of additional sequence dependent thermodynamic factors not explicitly considered by existing models. One of these is the potential influence of sequence context, beyond nearest-neighbor interactions, on duplex oligomer stability, which has not yet been considered in models aimed at predicting melting stability of short duplex DNA oligomers.

Evaluations of DNA sequence dependent thermodynamic stability have focused entirely on studies of the thermodynamic behaviors of solutions of homogeneous populations of individual molecules. Little attention has been given to the consideration of sequence context in multiplex hybridizations, where many component single strands are present in the same reactions. If their sequences are so predisposed these strands can anneal with other strands that are not fully complementary, to form partial duplex states that have enough favorable interactions to be relatively stable. Consideration of the stabilizing interactions that give rise to these cross-hybridizing states, and determining their relative stability and probability of occurrence is essential for accurate sequence design and selection in multiplex hybridization schema.

In general, there are two general scenarios wherein the analysis of nucleic acid hybridization is desirable. The first situation is in the design of a nucleic acid sequence that hybridizes selectively with a nucleic acid target. The second situation where nucleic acid hybridization analysis is desirable is in the design of a combination of unique nucleic acid sequences for general nucleic acid screening.

A method of analysis of nucleic acid sequences of the art is a program that can be used to generate useful oligonucleotide probes for a specific nucleic acid target. Such programs analyze and compare nucleic acid probe sequences for uniqueness including possible cross-hybridization with complementary or nearly complementary sequences, and they then estimate a melting temperature based on the number of GC and AT base pairs.

The “melting temperature” of a nucleic acid hybrid, or “Tₘ”, is the temperature at which 50% of a population of double-stranded nucleic acid molecules becomes dissociated into single strands. Equations for estimating the Tₘ of nucleic acid hybrids are well-known in the art. For example, the Tₘ of a hybrid nucleic acid can be estimated using a formula adopted from hybridization assays in 1 M Na+ and commonly used for calculating the Tₘ of PCR primers: (number of A+T)×2° C.+(number of G+C)×4° C. Newton et al. PCR, 2^{nd} Ed., Springer-Verlag (New York: 1997), p. 24. This formula, however, has been found to be inaccurate for primers longer that 20 nucleotides.

Other more sophisticated computations exist in the art which take structural as well as sequence characteristics into account in the calculation of Tₘ. A common approach is to consider the stacking interactions between each base pair in a hybrid with the base pairs on either side, which is known as “nearest-neighbor analysis”, to calculate Tₘ.

In practice, calculated melting temperatures are usually crude estimates; the results depend upon the parameters used, which can be inaccurate. Workers tend to use such crude estimates as a starting point for empirical observations of which probes are the best.

There are several technologies known in the art for microanalyses of nucleic acid hybridization on chips with arrays of nucleic acid probes. See, for example, U.S. Pat. No. 5,974,164, which discloses a computer-based method of selecting probes and designing the layout of an array of DNA or other polymers having certain beneficial characteristics. The generation of large numbers of useful probes, rather than mere starting points in amplification reactions, is increasingly important. Due to the large expense associated with carrying out the actual experiments, computer methods are important in the planning stages.

Sequence Context

The present invention provides a means for designing and selecting optimal oligomer sequences, such as those for use in multiplex array-based nucleic acid probe systems, down to the selection of a single pair of optimal primer/target oligomers. The methods employed specifically consider effects of sequence context on nucleic acid interactions. In some embodiments of the present invention, the methods of analyzing the potential interactions, e.g., hybridization, between nucleic acid sequences comprises the following steps.

At the heart of the analytical methods of the present invention is the representation of a pair of nucleic acid sequences, e.g., complementary pairs of sequences or non-complementary pairs of sequences, as a function characteristic of, and dependent on, the overall context of the sequences. Context is comprised of the sequence identity (i.e. A−T or G−C in the case of DNA), sequence order, and composition (% G−C). This is accomplished by representing each pair of sequences with a context functional descriptor, or CFD. The CFD integrates the features of sequence context into a functional representation of the sequences. This functional representation provides a method of analyzing, comparing, and selecting nucleic acid sequences. The CFD approach is employed because it can carry within it complete information about the context of each position of every base or base pair in a nucleic acid duplex. Context can be with regards to the whole sequence or certain windows of sequence at each base or base pair position along the entire sequence. In practice, the length of the window is commensurate with the use for which the nucleic acid sequence is intended, e.g., on a microarray.

Context information is encoded in the functional characteristics of the CFD. Molecular properties of nucleic acids, e.g., chemical structure, order of monomers in the sequence, and thermodynamic stability of base pairs, are captured in the CFD. Using sequence specific molecular parameters, e.g., measurements of base pairing and nearest-neighbor stacking interaction, to generate the CFD's provides physical meaning to each data point of the CFD. In addition, quantitative comparisons of CFD's for different pairs of nucleic acid sequences, e.g., a nucleic acid sequence and it perfect complement vs. the same nucleic acid sequence and a third nucleic acid sequence that differs from that of the perfect complement, enables comparisons of the context dependent components of the molecular properties of different pairs of nucleic acid sequences.

Any pair of sequences can be represented by a context functional descriptor (CFD). In a preferred embodiment, points on a CFD correspond to thermodynamic properties, e.g., ΔG, ΔH, ΔS, equilibrium constant, or tₘ, of the sequences and their context. In this functional representation duplex sequences can be analyzed and compared mathematically. As a result, higher quantitative significance can be given to sequence comparisons. This enables a more robust, complete and insightful method of sequence analysis and selection.

Even though the pervading influence of sequence context effects on oligomer hybridization is ever present, there currently are no analytical treatments that effectively consider effects of sequence context on duplex hybridization and thermodynamic stability. In addition, there are few practitioners of nucleic acid based amplification and detection assay methods that would argue with the assertion that; sequence context can and does influence primer/template binding and extension reactions and other hybridization reactions in ways that are not always reliable or predictable. From our perspective sequence context represents an essential component that must be considered in effective sequence design strategies.

The SEQ-TG™ technology provides an analytical framework for characterizing and evaluating sequence dependent context effects from a rigorous statistical thermodynamic basis. The SEQ-TG™ is both novel and broadly applicable to many different context dependent situations. Types of sequence context are defined in two ways depending on whether homogeneous or heterogeneous mixtures of strands are considered. Homogeneous mixtures are defined as those where only two strands are present with sequences that are complementary to one another. In this case, context refers to the explicit order and identity of all base pairs in the duplex state(s). In the case of heterogeneous mixtures, as occurs in multiplex reactions, where several or many different duplexes are present, context refers not only to the order and composition of base pairs in each perfect duplex, but also to the sequences and contexts of the other strands and their relative complementarities with respect to the perfect duplexes present.

Considering context effects represents a rather significant analytical challenge: to conceive of methods that consider long-range sequence dependent interactions. The methods must be general in that they need not be confined to order-dependent interactions, e.g., single base pair, nearest-neighbor, and next-nearest-neighbor interactions, as has been done in the past. In fact, it has been demonstrated that even under well controlled, high-resolution conditions, it is difficult to evaluate order dependent interactions to higher than nearest-neighbors. This does not mean that higher order sequence specific interactions do not occur or influence results, but simply that long range sequence dependent interactions at distances above nearest-neighbors are difficult to quantitatively dissect in a meaningful way by conventional methods.

The SEQ-TG™ design tool is founded on a new representation of DNA sequence. For each sequence, an ensemble of sequence specific configurations that depend explicitly on the identity and context of the entire sequence, contribute to the character of a so-called context function, the context functional descriptor (CFD). Each and every duplex sequence has a CFD. Using this approach effects of the entire sequence context are encoded for the sequence in the CFD. The actual form of the CFD need not be based on real chemical behavior of sequences. In principle, the CFD can have direct physical meaning or be completely arbitrary. The CFD merely serves as a means of coding context dependent sequence information in a consistent and useful functional form.

The Context Functional Descriptor (CFD)

The analytical approach employed in the present invention is based on using a novel “functional” representation of DNA sequence. This representation is termed the context functional descriptor, CFD. In principle, a CFD can be constructed for every combination of two strands that comprise a single duplex. For example, the duplex comprised of strands A and B has a CFD for all possible sequence pairs in anti-parallel orientation, i.e. A-B, A-A and B-B.

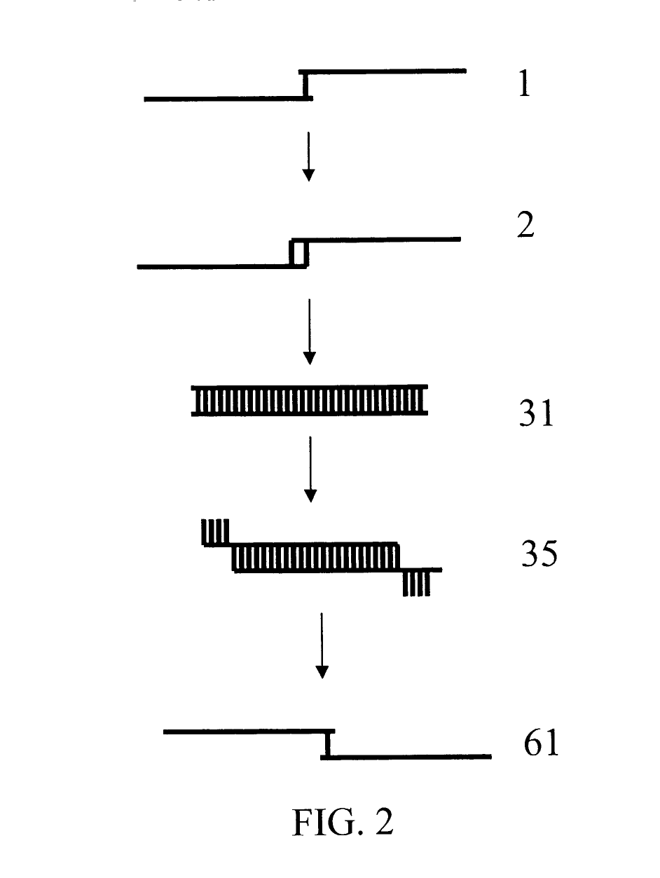

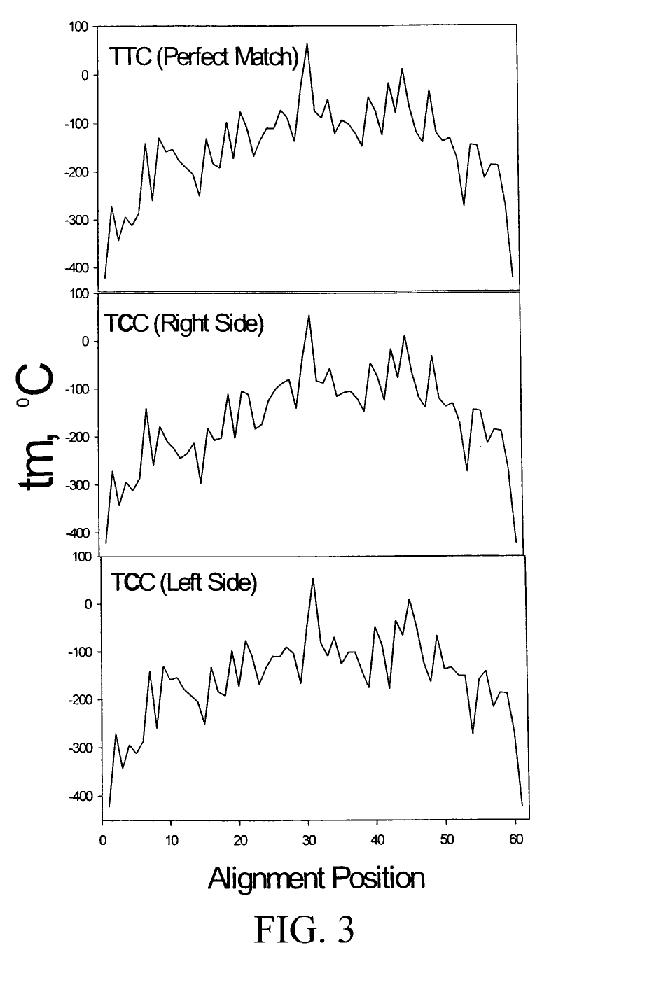

A CFD can, for example, be constructed by aligning two strands (e.g., 5′-3′/3′-5) and sliding one strand over the other, preferably one base at a time, and estimating experimental values for tₘ and/or thermodynamic parameters, such as ΔG, ΔH, or ΔS, for the hybrid duplex state at each alignment step. As discussed above, it is preferably done one base at a time to provide data for each position. However, fewer data points can be measured as long as the result is substantially the same. The sliding strand alignment scheme is depicted in FIG. 2A. One representation of a CFD is a plot of the estimated parameter(s) (e.g., tₘ, ΔG, ΔH, ΔS, or combinations thereof) for the hybrid duplex state at each alignment step versus the state, or alignment position, of the duplex (see, e.g., FIG. 3). The quantitative meaning of each point on the CFD depends on the parameters used to include effects of local sequence dependent interactions on the overall stability of the complex formed at each alignment position. In preferred embodiments, the numbers of aligned base pairs with corresponding hydrogen bonding contributions, nearest-neighbor dependent and next nearest-neighbor dependent stacking interactions, stabilizing interactions that occur when unmatched base pairs are shifted in the aligned sequences by one position to the right or to the left such that additional base-pairing occurs, and parameters for nearest-neighbor dependent single base pair mismatches are considered during construction of the CFD. The parameters employed can, for example, be taken from the literature. See, for example, SantaLucia, J. Jr., Proc. Natl. Acad. Sci. USA, 95:1460-1465 (1995); Owczarzy, R. et al. Biopolymers (Nucleic Acid Sciences) 52:29-56 (2000); SantaLucia, J. Jr., Biochemistry 26:9435-9444 (1998); SantaLucia, J. Jr., Biochemistry 8:2170-2179 (1998); and Hatim and SantaLucia, Jr., Biochemistry 34:10581-10594 (1997).

Using thermodynamic parameters to construct the CFD adds quantitative significance to the relative stability of each duplex considered. Presumably, some of these hybrid duplex states tolerate a number of base pair mismatches, and internal loops where multiple mismatched base pairs occur next to one another. The thermodynamic parameters, estimated for the hybrid duplex state of each alignment position, are represented by one or more points on the CFD. Over the course of considering all possible alignments and associated parameter values, the complete CFD is generated.

The complete CFD includes the estimated relative stability (tₘ) or thermodynamics (e.g., ΔG, ΔH, ΔS) of the alignments at every possible base position of the two strands. For every pair of single strand sequences aligned and compared in this way, there will be a corresponding CFD. For the two complementary strands that form a perfect matched duplex, the stability value of the perfectly base paired and stacked duplex corresponds to an extremum (maximum or minimum depending on the convention) on the CFD.

If tₘ is the quantitative calculated parameter, the tₘ of the perfectly matched duplex will be the maximum on the CFD. Alternatively, if the thermodynamic parameters, ΔG, ΔH, and/or ΔS, are the descriptive parameters estimated at each alignment position, the perfect duplex alignment would correspond to the extreme values (maximum or minimum depending on the standard state) on the CFD. In this way, instead of representing an N base pair duplex by a single point value (e.g., tₘ, ΔG, ΔH, ΔS), the N base pair duplex sequence is represented by a function comprised of 2N-1 points.

If the global minimum of thermodynamic parameters on the CFD corresponds to the perfectly aligned and base paired duplex, other local minima along the CFD correspond to partially base paired duplex configurations that can occur for particular sequence alignments. Using the thermodynamic parameters in this way, the deepest minima and highest maxima on the CFDs should necessarily correspond to the most and least stable partially base paired duplex complexes, respectively.

The shape of the CFD between these extreme points is a unique characteristic of the entire sequence context because construction of the CFD makes it strongly dependent on the actual base ordering frequency and content in the respective strands. As mentioned above, for each given pair of perfectly matched strands, the global minimum corresponds to the fully base paired duplex alignment. It should be noted that the value of the minimum for the perfect duplex is probably the most quantitative point on the CFD in the conventional sense, because the sequence dependent nearest-neighbor parameters are known with the highest quantitative accuracy for the perfect duplex state.

Parameter values estimated for the partial hybrid duplex states that occur in other alignment positions are based on assumptions about thermodynamic contributions of tandem mismatches and other structures to duplex stability. However, the impact of any possible uncertainty of this assumption is minimized since it is the relative differences of the CFD's in these regions that really matters when sequence are compared.

In addition, the stability of configurations in states with more than one mismatch in a row (referred to as tandem mismatch states) can be estimated from literature values for the sequence dependence of single base pair mismatches. Sequence dependent stabilizing interactions that might occur within such tandem mismatches can also be considered in constructing quantitatively meaningful CFD's. As constructed, the CFD of each duplex serves as a semi-quantitative functional signature of the relative stability of the ensemble of heteromorphic duplex states that can form between two strands. The particular shape of the CFD is explicitly dependent on sequence identity, composition and arrangement. That is, it depends on the overall sequence context.

Note that the extreme value for the perfect duplex is probably the most quantitative point on the CFD in the conventional sense, because the sequence dependent nearest-neighbor parameters are known with the highest quantitative accuracy for the perfect duplex state. In fact, when sequences are compared, it is the relative differences between the CFD's in these regions that are most relevant.

The particular form of the CFD described above was conceived from practical considerations. Other CFD's can also be envisioned. Sliding one strand over the other and constructing the CFD, with specific points corresponding to complementary sequences in partially overlapped alignments is a logical way to sample states that might occur during cross-hybridization. It was surmised that the partially aligned states for some sequences and their corresponding relative stabilities, compared to the perfectly matched duplex, could be an obvious source of cross-hybridization between strands present in multiplex reactions. Thus, for assessing the possibility for designing non-cross-hybridizing sequences for use in multiplex reactions, the CFDs have a direct physical interpretation. In essence, this is accomplished as follows. For every pair of sequences, the CFDs of each pair can be aligned at their extreme values. In this alignment, pairs of strands with quantitatively similar CFDs have similar sequence contexts, and therefore might be expected to have a stronger propensity for cross-hybridization.

Although nearest-neighbor parameters are available for many of the different types of single base mismatches, parameters for tandem mismatches where two or more mismatches occur next to one another, have not been evaluated. As part of the methods of the invention, the currently available n−n mismatch values were used, as described below, to estimate limits on the thermodynamic stability of states containing two or more tandem mismatches.

For the thermodynamic treatment of tandem (two or more) base pair mismatches, consider hybrid duplexes containing nucleic acid sequences that are not perfectly complementary (depicted in FIG. 2). The hybrid duplex states depicted have k mismatches sandwiched between intact pairs at positions j and j+k+1. Examples that are depicted are for k=5 and k=3. In this analysis, the enthalpic contribution (for example) of this local state (considering only the interactions directly involving mismatches) is given by,

ΔH_{L}=ΔH_{bp}(j)+ΔH_{bp}(j+k+1)+ΔH_{MM}(k) (1)

Where ΔH_{bp}(j) and ΔH_{bp}(j+k+1) are the enthalpic contributions from the intact base pairs at positions j and j+k+1, and ΔH_{MM}(k) is the enthalpic contribution from the k tandem mismatches. To estimate the value of ΔH_{MM}(k) the nearest-neighbor single base pair mismatch parameters can be used as follows,

$$Δ {H}_{MM} ( k ) = 1 / k {\sum }_{i = 1}^{k} Δ {H}_{j + i - 1 , j + i , j + i + 1} \qquad ( 2 )$$

The term in the sum ΔH_{j+i-1, j+i, j+i+1} is the nearest-neighbor dependent enthalpy for the single base pair mismatch at position j+1 with the specific neighboring base pairs at positions j+i−1 and j+i+1. In essence each mismatch in the tandem mismatch group can be treated as a single base pair mismatch and average over the nearest-neighbor dependent single base pair mismatch values for those sequences comprising the tandem mismatches. Since intact base pairs do not exist within tandem mismatches, using the single base pair mismatch parameters would be expected to overestimate the stability. This is because presumably stabilizing contributions of nearest-neighbor sequence dependent interactions of single base pair mismatches with neighboring intact base pairs should comprise a portion of the nearest-neighbor single base pair mismatch parameters. Actually, though, the presence of even additional stabilizing sequence dependent interactions within tandem mismatch groups must be considered to provide improved agreement with experimental observations for heteromorphic complexes. In fact, these stabilizing interactions add even more stabilizing influence than that contained in the mismatch pair to the calculated stability of the tandem mismatch loop. Because an additional stabilizing interaction must be included, the conventional estimates on stability contributions from tandem mismatches, as given in Eq. (2) for example, provide a lower limit estimate on the overall stability of tandem mismatch states. The requirement of these additional stabilizing interactions reveal that mismatch loops comprising tandem mismatched base pairs are fundamentally different from internal loops consisting of broken base pairs. In preferred embodiments of the present invention, this difference is explicitly considered (see below).

In preferred embodiments, the most current sequence dependent stability parameters, evaluated to the highest necessary order or interaction, are utilized in the estimates that make up a CFD. Parameters for nearest-neighbor base pairs and single base pair mismatches bounded by specific nearest-neighbors are utilized to make each point on the CFD as quantitative as possible. Here the sequence dependent parameters inherent in the Hyther™ program or recently reported nearest-neighbor parameters are used. See Owczarzy et al, Biopolymers (Nucleic Acid Sciences) 52:29-56 (2000); and SantaLucia and Peyret, HYTHER™ server—Department of Chemistry, Wayne State University.

In the nearest-neighbor model the enthalpy is written in terms of the hydrogen bonding component, ΔH_{H-bond}, that depends only on the number of A−T (T−A) and G−C (C−G), and the nearest-neighbor interaction component, ΔHₙ₋ₙ, determined according to,

ΔH_{duplex}=ΔH_{H-bond}+ΔHₙ₋ₙ=ΔS_{bp}[N_{AT}T_{AT}+N_{GC}T_{GC}]+ΣᵢⱼNᵢⱼ(δHᵢⱼ)+ΔH_{M} (3)

Where N_{AT} and N_{GC} are the numbers of A·T or G−C type base pairs in the duplex sequence. The average melting temperatures of A·T or G·C base pairs are given by T_{AT} or T_{GC}. The summed term in Eqn (3) includes the n−n sequence dependence. Nᵢⱼ is the number of times the n−n doublet ij (ij 1−10) occurs in the duplex sequence, and δHᵢⱼ is the deviation from the average nearest-neighbor dependent enthalpy for sequence doublet, ij. The final term, ΔH_{M}, accounts for single base pair mismatches or tandem mismatches, such as might occur in certain aligned states other than the perfect duplex. See Benight et al., Methods Enzymol. 340:165-92 (2001). Obviously, for the perfect duplex, ΔH_{M}=0. For single base pair, tandem and larger mismatches, ΔH_{M}=ΔHₘₘ+ΔH_{MM}, where ΔHₘₘ are the nearest-neighbor dependent single base pair mismatch parameters and ΔH_{MM} is calculated for tandem mismatches according to Eqn (2).

The entropy change of base pair melting, ΔS_{bp}, is assumed to be independent of sequence. In these calculations, the recently reported value for DNA oligomers at −22.4 cal/K.mol.bp is used. See SantaLucia, J. Jr. Proc. Natl. Acad. Sci. USA, 1998, 95, 1460-1465. The total transition entropy of the duplex is simply,

ΔS_{duplex}=ΔS_{bp}[N_{AT}+N_{CG}] (4a)

For aligned states with partial duplex overlap and single strand overhangs on the ends,

ΔS_{duplex}=ΔS_{bp}[N_{bp}+Nₗₒₒₚ] (4b)

where N_{bp} is the number of base pairs in the overlapped duplex regions and Nₗₒₒₚ is the number of single base pair, tandem base pair and larger mismatch loops.

The transition temperature, Tₘ, is calculated according to,

$${T}_{m} = ( Δ {H}_{duplex} + Δ {H}_{nuc} ) / ( Δ {S}_{duplex} + Δ {S}_{nuc} + R \cdot ln ( \frac{[ {C}_{T} ]}{4} ) ) \qquad ( 5 )$$

where ΔH_{nuc} and ΔS_{nuc} are the enthalpy and entropy of nucleation, respectively. A value of −9.0 cal/Kmol for ΔS_{nuc} can be employed. The nucleation enthalpy was determined according to,

ΔH_{nuc}=H₁−(H₂·f)−(H₃·Nₒᵥₑᵣₗₐₚ) (6)

where the values of H1, H2 and H3 are 7654.71, 3469.93 and 186.51, respectively, and the value off depends on whether the duplex is a perfect duplex or overlapped duplex. For a perfect duplex f=f_{GC}, the fraction of CG base pairs in the duplex. See Benight et al., Methods Enzymol. 340:165-92 (2001) and Owczarzy et al., Biopolymers 44:217-239 (1997).

For an overlapped duplex f=f_{bp}, the fraction of intact base pairs in the overlapped region. For a perfect duplex Nₒᵥₑᵣₗₐₚ is just the total number of base pairs. For other aligned states, Nₒᵥₑᵣₗₐₚ is the number of overlapped bases in each aligned configuration.

Regardless of the particular parameter sets that are employed to construct the CFD, every possible alignment of two strands is sampled. Because the value of each point depends on all of the other members of the sequence, the relative order and explicit identity of every base is a function of the entire sequence and its context. As it should, this functional representation of DNA sequence also incorporates (as a single point) the sequence dependent values for the perfectly aligned duplex. The calculated stability (e.g., tₘ, ΔH, etc.) determined in the conventional sense corresponds to this most extreme point on the CFD. Sequence dependent features of sequence order and composition or context, are contained in the actual shape of the CFD. Statistically significant populations of heteromorphic hybrid micro-states contribute, in a semi-quantitative sense, to the shape of the CFD. This added dimension provides an expanded representation of DNA sequences thereby providing a broader basis for making subtle sequence comparisons of stabilities and cross reactivities of different oligomeric sequences and their mixtures.

For the application of designing non-cross-hybridizing sequences for use in multiplex reaction environments, the CFDs employed in the examples have a direct physical interpretation. After alignment of their minima, pairs of sequences with similar CFDs have similar contexts and therefore a suspected propensity for cross-hybridization.

In contrast, application of the CFD for analysis of the sequence contexts of different homogeneous populations, each with a single duplex sequence, i.e. only two kinds of strands complementary to each other, resulting correlations of the CFD need not necessarily correspond directly to actual differences in the populations of partially paired duplexes in certain aligned states. In this case the CFD is merely a context dependent functional descriptor of sequence.

It bears repeating that in formulating the CFD of the examples full use is made of currently available nearest-neighbor dependent stability parameters for intact base pairs as well as single base pair mismatches. The very “best” available quantitative sequence dependent stability parameters are utilized to define each point on a CFD. Consequently, the CFD contains all the stability information in the sequence that can be calculated in the conventional sense, which corresponds to a single point, the global minimum on the CFD, and more.

So, in addition to the conventional characterization, important expanded information about the sequence order and composition of the context, that could actually correspond to statistically relevant micro-states are contained in the shape of the CFD. In effect our method provides a much richer representation of DNA sequences. In essence a duplex sequence is not represented by a single value (tₘ, ΔG, ΔH, ΔS), rather it is given by a quantitative function, the CFD.

Comparison of Nucleic Acid Sequences

In preferred embodiments, the methods of the present invention include the quantitative comparison of different perfect matched duplexes that may have different sequence contexts. The quantitative comparison of two duplexes comprised of perfectly matched strands, that may have different sequence contexts, can be comprised of the following steps: (1) Consider duplex A comprised of strands A1-A2, and duplex B comprised of strands B 1-B2. Strand A1 is perfectly complementary with strand A2 and strand B 1 is perfectly complementary with strand B2. (2) Represent the sequences as functions by constructing the CFD's for duplexes A and B, CFD_{A} and CFD_{B}. (3) Compare the two duplexes through mathematical comparison of CFD_{A} and CFD_{B}. For example, the correlation coefficient, the variance, or any quantitative method of scoring the functional similarity of CFD_{A} and CFD_{B} can be employed for this comparison. The degree of similarity between the two functions provides a quantitative determination of the similarity of duplexes A and B, which include different features of the respective sequence contexts of duplexes A and B. The character or shapes of CFD_{A} and CFD_{B} define the reference shapes of the perfect duplexes and represent the ideal context and most stable energetic environment for strands A1 and A2 in duplex A and strands B1 and B2 in duplex B. Since the CFD of a sequence provides detailed information about the context of the sequence, similarity between the CFDs of two sequences is an indication that the two sequences may have similar sequence-dependent properties. The converse in not always true, however: two sequences that have similar sequence-dependent properties need not have similar CFDs. In such cases, it may be possible to perform principle component analysis on the CFDs of the sequences so as to determine whether there is one or more component parts to the CFDs of the sequences that contribute their similar properties. Principle component analysis of CFDs is discussed further below.

In other preferred embodiments, the methods of the present invention include the quantitative comparison of perfect matched duplexes with imperfect match duplexes, e.g., those hybrid duplexes in which all bases on one strand are not perfectly matched with a complement on the other strand, e.g., the duplex contains mismatched base pairs and/or unpaired loops. The methods can include the following steps: (1) Consider the situation where both duplex A and duplex B and their constituent strands are present in the same solution. If their sequences are similar enough, the strands of duplex A could pair with those of duplex B, resulting in the possible hybrid duplex A-B, consisting of strands A1 and B2, and B-A, consisting of strands B 1 and A2, as well as other possibilities. (2) Construct the functional representation (the CFD's) for the hybrid duplexes, CFD_{A-B} and CFD_{B-A}, and quantitatively compare their shapes with the reference CFD_{A} and CFD_{B}, respectively. The correlation coefficient, variance, or any quantitative method of scoring the functional similarity of CFD_{A} and CFD_{A-B} or CFD_{B} and CFD_{B-A} can be employed for this comparison. The degree of similarity of the two functions provides a quantitative determination of the similarity of the hybrid duplexes A-B and B-A with the reference duplexes A and B, respectively. In some preferred embodiments, the comparison involves first aligning the extreme points, e.g., maxima or minima (depending upon the type of data present in the CFD, e.g., tₘ vs. ΔG, of the CFDs of the reference duplexes and the hybrid duplexes. In cases where the extreme points of the CFDs are not in the same position, such that the CFDs are not fully aligned when the extreme points are lined up, unaligned portions of the CFDs can, for example, be brought into alignment by shifting the unaligned data of one of the CFDs from one end to the other. The degree of quantitative similarity observed between two CFDs defines the cross-hybridization propensity of the corresponding nucleic acid sequences, in this case A and B.

Principal Component Analysis and Prediction of Nucleic Acid Properties

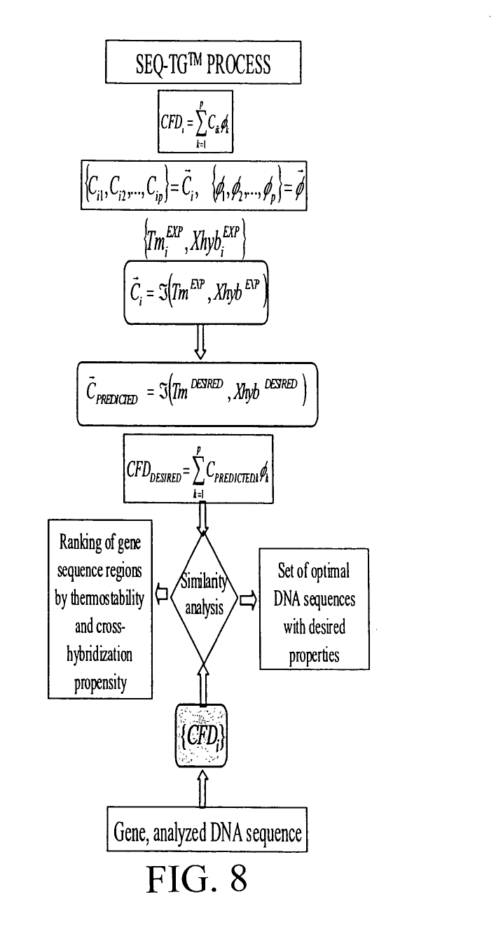

Using a representative set of sequences and experimental results, it is possible to relate experimental tₘ's or some other property of a nucleic acid sequence (or even a peptide encoded therein) with certain one or more principle components of a sequence's CFD. The resulting information can then be employed to generate or identify sequences that have similar, or even superior, properties, e.g., by following the general procedure diagrammed in FIG. 8. The methods are as follows:

(1) perform principal component analysis on the CFDs of sequences having properties of particular interest. The CFDs are deconstructed into linear combinations of a minimal number of basis CFD's. Principal component analysis reduces the necessary sampling of the experimental space and increases the statistical robustness of the relationships that are employed. The result is a minimal set of common CFD basis functions, φₖ, and sets of coefficients (loadings), Cᵢₖ, that reproduce the individual CFDᵢ from the CFD basis functions.

(2) find functional relationships ( [symbol] ) between experimentally measured properties of interest, e.g., tₘ's (Tₘ^{EXP}) and cross-hybridization propensity (Xhybᵢ^{EXP}), and the loadings determined in step (1), e.g., Ci= [symbol] (Tm^{EXP}, Xhyb^{EXP}).

(3) employ the resulting functional relationship to predict the loadings for any desired Tmᵢ^{EXP} and Xhybᵢ^{EXP} within a desired interval range, C_{PREDICTED}= [symbol] (Tm^{DESIRED}, Xhyb^{DESIRED}) A number of methods (from artificial intelligence and chemometrics arsenals) are available for this task. See Simpson, P. F. Artificial Neural Systems, Pergamon Press (New York: 1990); Matthias, O. Chemometrics: Statistics and Computer Applications in Analytical Chemistry, Wiley-VCH (Weinheim, N.Y.: 1999); Gardiner, W. P. “Statistical Analysis Methods for Chemists: A Software Based Approach,” Royal Society of Chemistry, Cambridge (1997); and Wasilevsky, A., “Statistical Factor Analysis and Related Methods; Theory and Applications, Wiley Series in Probability and Mathematical Statistics,” J. Wiley (New York: 1994).

(4) use the basis CFD's and predicted loadings to generate the shape of the desired CFD for any sequence with the user defined desired properties (e.g., Tₘ and cross-hybridization propensity).

(5) for the sequences to be analyzed, generate all CFD's of all possible n-mer sequences.

(6) perform quantitative similarity analysis of the constructed CFD's with the desired CFD. See Matthias, O., Chemometrics: Statistics and Computer Applications in Analytical Chemistry, Wiley-VCH, Weinheim, N.Y.: 1999.

Sequences arrived at in this manner are those that have the highest similarity with the desired CFD and thus should display optimal properties with respect to user defined desired properties. With this process the SEQ-TG™ technology provides a more rational and quantitative and reliable approach to sequence design and engineering. Any specified target sequences can be used as input, and the SEQ-TG™ will provide the most compatible sets of oligomers, where compatibility of sequences is strictly defined as those sequences that are isothermal and non-cross-hybridizing, or other desired sequence dependent properties.

In summary, the SEQ-TG™ technology is founded on a rigorous statistical thermodynamic basis, coupled with a novel approach for representing sequences and their full contexts. This collective approach provides a sequence design tool for which there is currently no commercially available counterpart.

Selection of Optimum Sequences

The present invention provides methods for utilizing duplex stability and sequence context information to systematically compare the members of a set of nucleic acid sequences and thereby identify subsets of nucleic acid sequences having desired tₘ's and cross-hybridization propensities. Methods of the invention are useful for selecting optimum sets of nucleic acid probes wherein the probes are diagnostic with respect to a particular set of target sequences, e.g., naturally occurring nucleic acid sequences (e.g., fragments of a genome, cDNA library, or alternatively spliced exons, or collection of SNPs or mutational hot spots) or a set of generated sequences (e.g., generated computationally), and wherein the probes do not display appreciable cross-hybridization with, e.g., the targets of other probes.

In preferred embodiments, the methods of the invention include the following steps:

obtaining a set of nucleic acid sequences each having a length N (typically, N=5 to 150);

sorting the nucleic acid sequences in the set into isothermal groups according to their calculated tₘ's (for a perfect match duplex) and identifying an isothermal subset of the sequences wherein each sequence in the subset has a predicted tₘ approximately equal to a target temperature, e.g., the tₘ's are all within a +/−2° C. interval of the target temperature;

determining, within the isothermal subset of nucleic acid sequences, the quantitative similarity descriptors (e.g., correlation coefficients, variance, or some combination thereof) between the CFD of each nucleic acid sequence (and it perfect complement) and the CFDs of the nucleic acid sequence with each complement of the nucleic acid sequences of the isothermal subset;

using a threshold value of the quantitative similarity descriptors (e.g., correlation coefficients, variance, or some combination thereof) of each nucleic acid sequence in the isothermal set to score the nucleic acid sequence for its propensity to cross-hybridize with each complement of the other nucleic acid sequences in the isothermal set; and

identifying a third set (a subset of the isothermal set) of nucleic acid sequences having the properties that the nucleic acid sequences of the third set are isothermal and non-cross-hybridizing with all of the complements of the other nucleic acid sequences in the third set.

In some embodiments, obtaining a set of nucleic acid sequences each having a length N (typically, N=5 to 150) includes constructing the set from target sequences, e.g., naturally occurring sequences, e.g., genomic DNA or cDNA library sequences, by sliding a window of N bases over the target sequences, e.g., one base at a time. For genomic DNA, the size of the resulting set of nucleic acid sequences may be too large to analyze in a reasonable amount of time. Therefore, it may be useful to perform the analysis by starting one gene at a time, and then pooling all of the non-cross-hybridizing sequences from each gene in the genome and performing the selection analysis on the resulting set of sequences to thereby identify sequences from throughout the genome that are non-cross-hybridizing with one another.

In some embodiments, the tₘ target temperature used to define the isothermal subset is selected so as to maximize the number of sequences present in the isothermal subset. It is desirable to maximize the number of sequences present in the isothermal subset so as to maximize the number of sequences present in the isothermal, non-cross-hybridizing, third set of nucleic acid sequences. In other embodiments, the tₘ target temperature used to define the isothermal subset is selected for reasons other than maximizing the number of sequences present in the isothermal subset, e.g., for reasons related to the intended use of the nucleic acid sequences. It is also possible to increase the number of sequences present in the isothermal subset by increasing the temperature interval, e.g., from +/−2° C. to +/−3° C. However, increasing the temperature interval can also reduce the performance of the nucleic acid sequences when used as a set, e.g., on a microarray.

In some preferred embodiments, the quantitative similarity descriptor is two-dimensional and has (1) a stability coordinate defined by

$${( \frac{σ}{{Norm}_{σ}} )}^{x} {( {r}_{ij} )}^{y}$$

where “i” is a nucleic acid sequence, “i′” is the perfect complement of i, “j′” is a nucleic acid sequence that differs from i′, rᵢⱼ is the correlation coefficient, σ is the variance, Norm_{σ} is a normalization factor, and x=4 and y=6 (although x and y can be varied depending upon performance requirements), and (2) a context coordinate defined by:

$$( \frac{ΔΔ {H}_{ij}}{{Norm}_{ΔΔ H}} ) ( \frac{m}{overlap} )$$

where ΔΔHᵢⱼ is the difference in the minimum change in entropy of the duplex ii′ and the minimum change in entropy of the duplex ij′, Norm_{ΔΔH} is a normalization factor, m is the number of stabilizing interactions in the overlapping minimum energy state for the duplex ij′, and “overlap” is the total number of bases in the overlapping hybridized section of the duplex ij′. Non-cross-hybridizing sequences can be defined, for example, as those sequences having a stability coordinate >=0.4 and a context coordinate <=1.3. However, as with the x and y parameters, the threshold values for the stability and context coordinates can be varied depending upon performance requirements.

In some embodiments, experimental results involving some of the nucleic acid sequences of the set are used to calibrate the threshold value(s) for the quantitative similarity descriptors, e.g., correlation coefficients, variance, or any combination thereof. For example, if the cross correlation coefficient is found to be 0.6 for a particular hybrid duplex compared to the corresponding perfect match duplex, but the experiment of that hybrid duplex does not reveal a melting transition (no cross hybridization observed) in solution experiments, the cross-hybridization threshold would be assumed to be above 0.6.

In some embodiments, the nucleic acid sequences are ranked according to their predicted stability (for the perfectly complementary duplex) and cross-hybridization propensity (determined, e.g., from the minima and correlation coefficients of their CFD's with the global minima aligned with respect the perfect match CFD's), and this ranking is used to select the third set of isothermal, non-cross-hybridizing nucleic acid sequences.

In other embodiments, the isothermal, non-cross-hybridizing third set of nucleic acid sequences is selected using a streamlined mathematical technique for identifying a clique (in this case, a set of nucleic acid molecules in which all member of the set are isothermal and non-cross-hybridizing with the complements of the other nucleic acid sequences in the set). The technique can include:

creating an M×M matrix, wherein M is the number of nucleic acid sequences in the isothermal set and each entry in the matrix, Sᵢⱼ, contains the quantitative similarity descriptor for the CFD of the duplex ii′ as compared to the CFD of the duplex ij′ (wherein i and j are nucleic acid molecules of the isothermal set, and i′ and j′ are the perfect complements of i and j, respectively);

reassigning the Sᵢⱼ values in the matrix according to the threshold conditions for cross-hybridization such that Sᵢⱼ=1 for nucleic acid molecules that are predicted to be non-cross-hybridizing and Sᵢⱼ=0 for nucleic acid molecules that are predicted to cross-hybridize;

rearranging the rows and columns of the matrix so as to identify a submatrix containing only Sᵢⱼ=1 values, thereby defining a core set of non-cross-hybridizing sequences; and

performing pair-wise comparisons of each sequence outside the core with sequences in the core and adding to the core any sequence that is non-cross-hybridizing with all of the sequences of the core, thereby selecting an isothermal, non-crosshybridizing third set of nucleic acid sequences.

Rearranging the rows and columns of a matrix so as to identify the largest submatrix (the “clique”) containing only Sᵢⱼ=1 values is computationally intensive. Consequently, the present method uses a “crystallization” technique, wherein the algorithm for rearranging the rows and columns is run for a specified amount of time and the largest submatrix containing only Sᵢⱼ=1 values at the end of that time is defined as the core. This method produces a reasonably large set of isothermal, non-cross-hybridizing nucleic acid sequences, but it does not produce a unique core, as the order in which additional sequences are entered into the core (by pair-wise analysis with all of the sequences in the core) influences the final result. Furthermore, more than one “crystal” (a set of sequences in which the sequences are all non-cross-hybridizing with the complements of the other sequences in the set) can be identified in a set of isothermal sequences, each of which can be used to produce an isothermal, non-cross-hybridizing set of nucleic acid sequences. Thus, both overlapping and non-overlapping sets of isothermal, non-cross-hybridizing nucleic acid sequences can be identified.

As an example, to determine the similarity in a set of M sequences, an M×M matrix can be constructed wherein the row elements are the M sequences and the column elements represent the respective perfect complements of the M sequences. To compute the matrix, the CFD for each duplex formed by element i (i-th sequence) and element j-th complement) is determined. In parallel, another matrix is computed, rij, whose elements are

rᵢⱼ=Integrate(CFD_{ii′}−CFDᵢⱼ)/MᵢᵢMᵢⱼ

The values of rᵢⱼ are between −1 and 1, with 0 indicating total difference, and 1 indicating total similarity.

The core of the program is a routine energy function, which computes enthalpy, the energy given by mismatches, and the melting temperature. Computations are undergone with respect to nearest-neighbor theory:

$$\begin{matrix}Δ {H}_{duplex} = {k}_{singlet} Δ {H}_{singlet} + {k}_{nucl} Δ {H}_{nucl} + {k}_{nn} Δ {H}_{nn} ++ \\ {k}_{nnn} Δ {H}_{nnn} + {k}_{mm} Δ {H}_{mm} + {k}_{loop} Δ {H}_{ATloop} ++ \\ {k}_{loop} Δ {H}_{CGloop} \\ {T}_{m} = Δ {H}_{duplem} / Δ {S}_{duplem} + {R}_{log ( CTOT / α )}\end{matrix}$$

The energy of a duplex originates from several contributions:

a) Nucleation energy: this part of the energy comes from formation of a nucleation core. Empirically it is known that the presence of CG pairs in the core is crucial. So evaluation was formerly based on content of CG pairs in duplex area. This lead to faulty behavior in duplexes with small number of matched pairs, if CG pair was present. Thus, it is presently being evaluated from a ratio of matched pairs to the length of sequence.

b) Hydrogen bond energy: number of matched pairs is determined in the duplex area, and experimental values are assigned separately for CG and AT pairs.

c) Nearest neighbor energy: if two matched pairs are neighboring, experimental value to such arrangement of four bases is obtained for example from the prior art and added.

d) Next nearest neighbor energy if three matched pairs are neighboring, experimental value to such arrangement of four bases is obtained, for example, from the prior art and added.

e) Mismatch energy: experimental values are known only for simple mismatches. In such cases the contribution is an average of fours made with left neighbor of mismatched pair and of four with right one. If several mismatches occur subsequently, then for each of them one base is changed in a neighboring pair to achieve a match. These changes always prefer GC pair, so if it is possible to form one, it is formed.

f) Loop energy: if there are at least two neighboring mismatches, there may be a possibility of forming a match between two bases in positions shifted by one register. A base not involved in match interaction can take place in loop

Design and Generation of Sequences with Predefined Properties

For the purposes of this aspect of the invention, nucleic acid sequences are classified as being of two general types: natural or synthetic. Natural sequences are of natural origin, exist in nature, and comprise, e.g., genomes, cDNAs, SNPs, etc. Synthetic sequences are nucleic acid sequences that have been generated, e.g., computationally, and need not have a natural counterpart. Synthetic nucleic acid seuqences can be used, for example, as tags for a universal microarray.

The methods of the present invention are useful for generating synthetic nucleic acid sequences. Nucleic acid sequences of length N (N=2 to b) are built from the set of possible nucleotide base monomer units, e.g., A, G, C, T, and/or any other base monomer to have predefined composition and properties.

In some embodiments, the methods include:

specifying the sequence length and, optionally, the desired % G−C;

determining one or more base compositions, e.g., numbers of A, T, C, and G bases, of the synthetic nucleic acid sequences that satisfy the sequence length condition and, if applicable, the % G−C condition;

providing, for each base composition, a partial representation, e.g., a partial mathematical representation, e.g., an incomplete sequence graph or n×n matrix (where n is the number of different types of bases in the nucleic acid sequence, e.g., n=4 for DNA), corresponding to a set of synthetic nucleic acid sequences that have the same base composition;

partitioning, for each base composition (or partial representation), the bases, e.g., A, T, G and C, into many different, e.g., all possible, nearest neighbor connections that satisfy the sequence length and base composition conditions, thereby providing for each partial representation a set of complete representations, each of which corresponds to an isothermal (within the limits of the nearest-neighbor approximations) set of nucleic acid sequences; and

enumerating all of the isothermal nucleic acid sequences defined by each complete representation, thereby generating a set of synthetic nucleic acid sequences.

In preferred embodiments, the nucleic acid sequence length is about 15 to 100 bases, more preferably about 20 to 80 bases, and most preferably about 25 to 60 bases.

In preferred embodiments, the GC content (% G−C) of the nucleic acid sequences is 50% +/−20%. In other preferred embodiments, the G and C content of the nucleic acid sequences is each 25% +/−10%. In still other preferred embodiments, the A, T, G, and C content of the nucleic acid sequences is each 25% +/−10%.

In preferred embodiments, all of the possible base compositions that satisfy the sequence length and base composition conditions, e.g., % G−C, G and C composition, or A, T, G, and C composition, are determined.

In preferred embodiments, the representation of base composition is a n×n matrix (wherein n corresponds to the number of different bases that are included in the nucleic acid sequences) or a sequence graph that is Eulerian. In particularly preferred embodiments, the representation of base composition is a 4×4 Eulerian matrix, e.g., as described herein.

In preferred embodiments, the partitioning of the bases with respect to nearest-neighbor connections is performed in all possible ways such that all possible distributions of nearest-neighbor connections are sampled. It is understood that each unique distribution of nearest-neighbor connections for a given sequence length and composition will have a unique representation, e.g., a unique 4×4 Eulerian matrix representation.

In preferred embodiments, the complete nucleic acid sequence representations are enumerated, in part, by determining the basic sequence cycle compositions of the sequence representations. For matrices, this can be accomplished using linear algebra and the matrix equivalents of the basic sequence cycles, as defined below. Similarly, sequence graphs can be decomposed by systematically subtracting out basic sequence cycles. The basic sequence cycles can then be joined at their vertices (there are many different permutations for how the basic sequence cycles can be joined) and sequences extracted from the resulting graphs. This method is discussed below in the section describing Permutable Sequence Units.

In other embodiments, instead of starting from an adjacency matrix, the process of generating nucleic acid sequences can start from a cycle coefficient vector. Thermodynamically, the solution is the same—the cycle coefficient vector defines the same set of isothermal sequences as the sequence graph and its adjacency matrix. The anticipated advantage of this modification is that it will be possible to program this differently from the algorithm using the adjacency matrix. The most important difference is that new implementation techniques will allow the creation of sequence sets incident with cycle coefficient vector input one set at the time. Adjacency matrix-based programs have to keep all structures for generating of permutable units simultaneously in memory. The implementation of cycle coefficient vectors imposes much smaller memory requirements, will be capable to create one complete sequence set and store it. Thus, the limitations on the size of the generated sequence set will mainly be associated with computational time and R/W speed/capacity. The most important use of this modification will be for long sequences.

In another embodiment, the algorithm for sequence enumeration from the complete nucleic acid sequence representation can be as follows: (a) start with a sequence graph that contain a vertex E (representing the ends of the nucleic acid sequence) and beginning at vertex E, connect the out-port with the oriented edge to the in-port of vertex X that has at least one in-port; (b) form the oriented edge from the out-port of vertex X to the in-port of vertex Y that has at least one in-port; and (c) repeat steps a and b until all possible combinations of allowed connections between in and out ports are sampled.

In yet another embodiment, the algorithm for sequence enumeration from the complete nucleic acid sequence representation can be as follows: (a) start with a sequence graph that contain a vertex E (structurally the in-port of the E vertex represents the 3′ end of the sequence, the out-port represents the 5′ end of the sequence) and find all in-trees rooted in vertex E, which can be accomplished by methods known in the art of Graph Theory; (b) for each tree connect the vertex next to root E via its out-port to the next available in-port which is not part of the tree; (c) continue until all combinations of vertices with available in-ports are sampled. Use the vertices of the tree only if no other out-port is available. This generates all Eulerian graphs.

In another aspect, the method of generating synthetic nucleic acid sequences can be performed as described above with the exception that the nucleic acid base identities are not assigned until after sequences have been enumerated from the complete nucleic acid representations. For example, the bases can be represented generically as i, j, k, and 1. After enumerating the sequences from the complete nucleic acid representations, the actual bases, e.g., A, T, C, and G, can be assigned through permutations of the identities of i, j, k, and 1. This method allows a substantial reduction in computational processing time as it reduces the number of possible sequence compositions, partial representations, and complete representations being processed.

The potential for cross-hybridization of sequences generated by these methods can be determined using context functional descriptors and the methods described above.

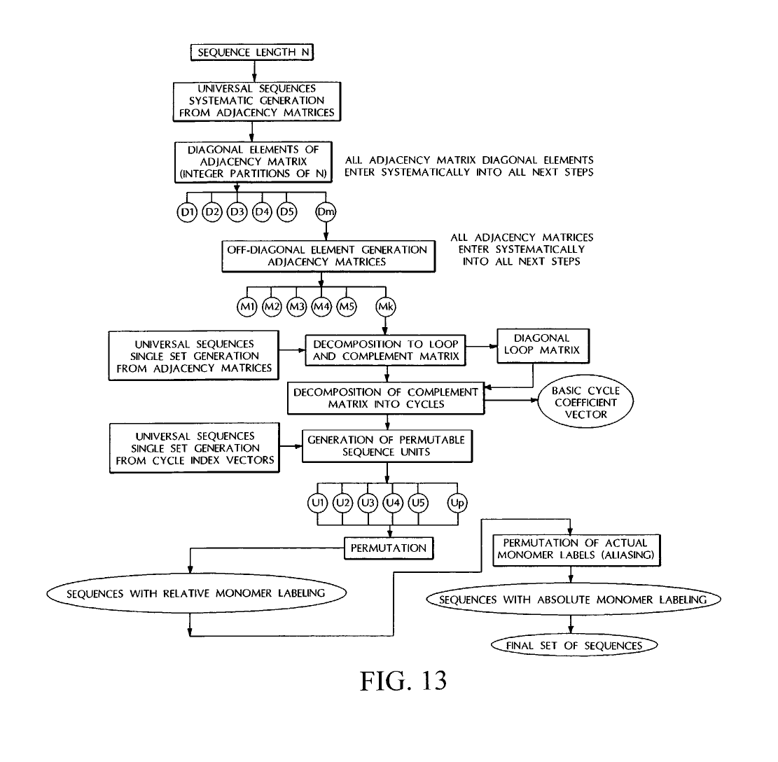

SEQ-TG™ can be used for sequence generation (see flowchart in FIG. 13). The algorithm will generate nucleic acid sequences of length N, without restrictions on the sequence primary structure other than the length, which is the input into the algorithm. A condition that is assumed implicitly is that all four bases are used.

N is integer number of bases in the desired sequence. The SEQ-TG™ algorithm uses adjacency matrix representation of the sequence graph to proceed and generate series of isothermal non-crosshybridizing sequences of length N. From this perspective, N is the common trace of a whole series of adjacency matrices. To find individual adjacency matrices from this series, all partitions of N into four numbers (diagonal elements of the adjacency matrix) are found first and stored in memory. For longer sequences (for example, for 70-mers, there are 52,594 partitions of N=70 into four diagonal elements; for 100-mers there are 113,564 of them, etc.) the number of partitions should be reduced to ensure that the algorithm can proceed in real time and within memory capacity.

The first reduction is achieved by adopting relative labeling of adjacency matrix columns (sequence graph vertices). With this convention, it is not necessary to consider all twenty-four permutations of A, T C and G over the numbers for each partition. The same reduction is experienced in all subsequent steps of the SEQ-TG™ algorithm. The change of sequences with relative labeling of monomers into real sequences with the identities of bases explicitly given is done before the calculation of the context functional descriptors. Software implementation of this step realizes systematic permutation of actual base identities for each relative label a, b, c and d. This step is straightforward and fast.

Further reduction is implemented so that a maximal number of non-crosshybridizing sequences can be obtained in the final output of the algorithm. Molecularly, this goal will be most likely achieved with sequences that have a balanced fractional composition of bases (for DNA, about 25% of each base). This composition provides the maximal number of variable positions for individual bases in the sequence, thus providing maximal propensity for mismatches if different sequences of the same set are interacting. The algorithm for partitioning N into diagonal elements allows a range around 25% for each base and only diagonal elements within that prescribed fraction range are further processed. Depending upon the hardware, a typical number of unique adjacency matrix diagonals created in this initial step is about 1000.

In the next step, each diagonal is expanded into off-diagonal elements, thereby generating complete adjacency matrices corresponding to sequence graphs. This is, again, a partitioning of the integer value of diagonal element into three numbers. The partition is nevertheless restricted by conditions given by the molecular structure of the DNA sequence. Therefore, this partitioning proceeds as follows.

The first step allows for the possible existence of sequence motifs having adjacent bases of the same type (say ˜AAA˜ etc.) in the final sequence. These motifs are represented by loops in the sequence graphs and in the corresponding adjacency matrices the number of these loops is given by the difference between the diagonal element and the sum of row or column off-diagonal elements incident with that diagonal element. The adjacency matrix can be decomposed into a sum of two matrices—square A1 and diagonal A2—as discussed below (see Matrix Representation of Sequence Graphs). The first step of the diagonal element partition is a reverse of this process—diagonal element D is systematically reduced by d=1, 2, . . . , D−1, D. The value d defines the corresponding diagonal element of A2 and the diagonal element in matrix Al is defined as (D−d). When this is done for all diagonal elements, matrix A2 is fully determined (it is a diagonal matrix).

Among other things, these methods allow practical and insightful generation of probe/target pairs for use on microarrays, and in PCR and cloning systems. By design, selected sequence pairs will have the properties of being isothermal, non-cross hybridizing or cross-reacting, and give more uniform and sensitive results (for example, uniform fluorescence intensities on a microarray).

These methods will enable the enumeration of the possible microstates thereby providing for more accurate predictions, enhanced modeling capability and predictive power of non-two state behavior on two dimensional microarray surfaces such as biochips, slides or beads.

A direct application of the methods and tools of the present invention is in quantitative sequence design of multiplex hybridization reactions.

Because the present invention provides a quantitative understanding of cross-hybridization in multiplex environments and thereby it allows for increases in the number of sequences that can feasibly be employed in multiplex hybridization reactions. This enablement derives from the understanding of the molecular states and corresponding sequence dependent tolerance levels for cross hybridization. If cross hybridization reactions are quantitatively understood, it is possible to employ cross hybridization levels as useful diagnostic indicators and it is possible to provide a quantitative understanding of this behavior. From this we are able to define optimal probe/target pairs and primers for use in better quantitatively querying genomic sequences for purposes of expression analysis, SNP detection etc.

The present invention provides tools to locate functional regions of any genome (see claims). Such an embodiment preferably begins with a sequence description of a consensus functional region. Using a method of the present invention, nucleic acid sequences are selected that are useful for uniquely identifying a sequence in agreement with the consensus functional region. Then those sequences are used to search a genome for the selected unique sequences or their complementary sequences.

The present invention provides quantitative parameters of sequence-dependent properties of genomic sequences that can be used in any quantitative structure/property relationship algorithm.

The present invention provides an analytical method for characterizing and finding special sequence motifs in large genomes such as binding sites for small ligands and proteins.

An advantage of using methods and tools of the present invention is that with this approach, higher order dependent interaction need not be explicitly known, but can be treated quantitatively.

Mathematical Descriptors of Nucleic Acid Sequence

Sequence Graphs

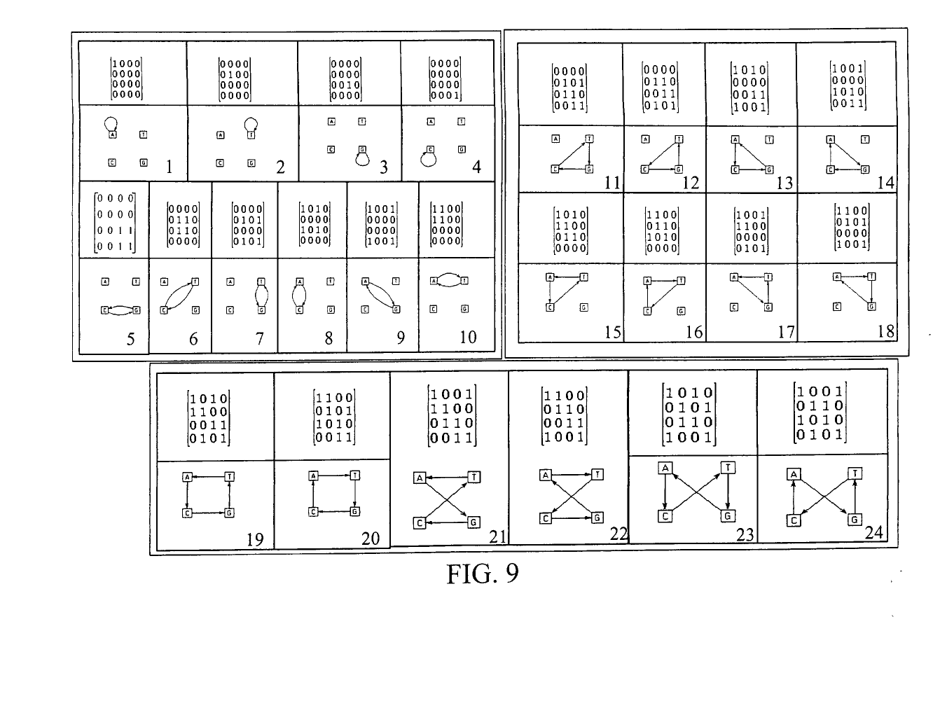

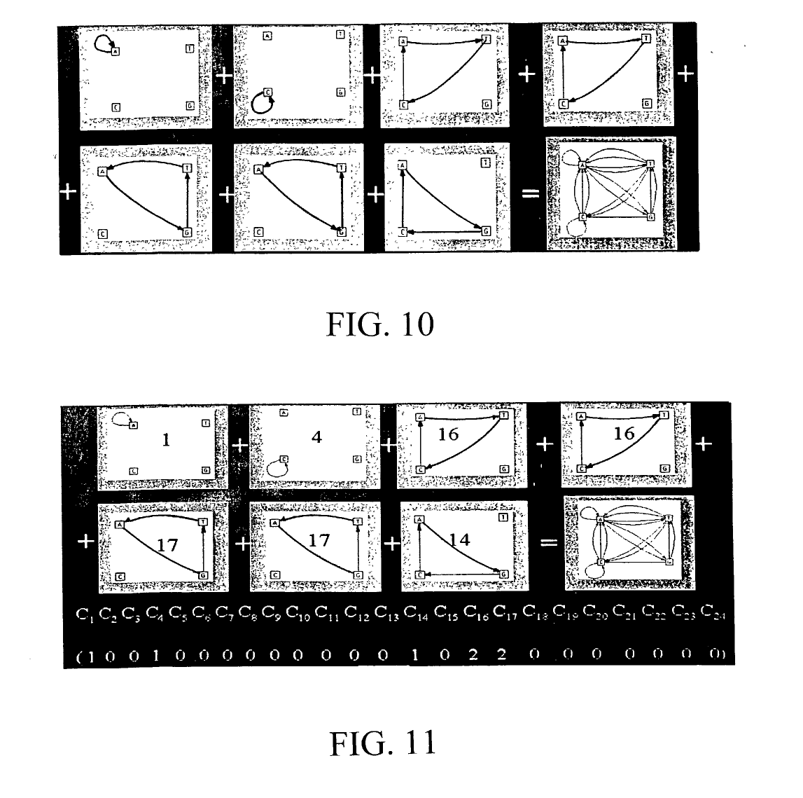

Sequence graphs are composed of vertices and edges that link the vertices. Typically, sequence graphs have as many vertices as there are monomeric units from which the biopolymer being represented is composed. Thus, sequence graphs for DNA molecules have four vertices (see FIGS. 9 and 10). These vertices can either be labeled by the respective monomer identities (A, T, C and G for DNA) or can be labeled only relatively (e.g., i, j, k and 1 for DNA) with labels representing only relative difference in monomer composition. Relative labeling is very useful for minimizing of the complexity of algorithms, e.g., SEQ-TG™ algorithms, which can work in one pass with the relative vertex labeling, only assigning monomer unit identity once on the algorithm is complete. At that point, labels are assigned systematically for all permutations of the monomeric units (e.g., for DNA labels a, b, c and d are filled with all permutations of A, T, C and G). In some cases, sequence graphs include an additional vertex, E, which denotes the ends of the polymer.

In molecular terms, the edges in sequence graphs represent covalent links between the monomer units represented by the graph vertices. Sequence graphs can also have loops, edges that start and end in the same vertex. Thermodynamically, the edge in a sequence graph represents the contribution of nearest neighbor interactions (primarily stacking interactions in the case of DNA) between two monomer units (represented by the graph vertices) to the overall stability of the polymer.

The basic molecular feature of nucleic acid molecules—that they are linear non-branched polymers of monomeric units—determines that the sequence graph is Eulerian. Thus, specific properties of the sequence graph are captured in mathematical theorems for Eulerian graphs. In particular, the mathematical properties of Eulerian graphs provide for a) a series of boundary conditions (which are essential in the SEQ-TG™ algorithms; and b) a unique decomposition of the sequence graph into basic cycles.

Matrix Representations of Sequence Graphs

Each sequence graph can be uniquely and unequivocally represented by, e.g., computer readable adjacency matrices or connectivity tables. Using adjacency matrices to represent sequence graphs involves a square matrix wherein the number of rows is identical to the number of monomeric units from which the biopolymer sequence is synthesized (for DNA, a 4×4 matrix is used). The rows and columns of adjacency matrices that correspond to sequence graphs are labeled by the chemical identities of monomeric units. This labeling can be direct or relative, in the same way as was shown above for sequence graph vertices. Entries on the main diagonal of the matrix represent the numbers of respective monomeric units in the sequence. The trace of the matrix corresponding to a sequence graph defines the total length of the biopolymer. Off-diagonal elements in the matrix indicate how many monomer units of type a and b (a and b referring to the labels of a particular row and column in the matrix) are connected by a covalent bond in the biopolymer primary sequence. The matrix representation of a sequence graph can be decomposed into a diagonal matrix representing the loops in the sequence graph and the residual sequence matrix. The residual sequence matrix has a unique property stemming from the fact that the sequence graph is Eulerian—each element of the main diagonal should equal the sum of the off-diagonal elements in both the row and the column in which the element belongs. By changing the sign of the off-diagonal element values to negative, the residual sequence matrix becomes a Laplacian matrix of the corresponding (residual) subgraph. Laplacian matrices allow the determine of the exact number of actual sequences associated with a given sequence graph. See Matousek and Nesetril, in “Invitation to Discrete Mathematics”, Oxford University press, Oxford, (1998); Chung, F. R. K. Spectral Graph Theory. Providence, RI: Amer. Math. Soc., (1997); and Bendito et al. “Shortest Paths in Distance-Regular Graphs.” Europ. J. Combin. 21: 153-166 (2000). Nonzero values of the determinant of a Laplacian matrix derived from a sequence graph indicate that the corresponding sequence graph is connected. Because all sequences incident with a given sequence graph will have the same length and fractional monomer unit composition (a measure of the hydrogen bonding contribution to duplex stability), as well as the same number of nearest-neighbor (base stacking) interactions in their primary sequence, they are predicted to be thermodynamically iso-energetic up to the level of considering nearest-neighbor contributions.

Basic Cycles of Sequence Graphs

The fact that a sequence graph is Eulerian ensures that it can be drawn by a single path through all of the graph vertices, and include all of the edges of the sequence graph exactly once. There are many such paths associated with a single sequence graph. Every such path represents one biopolymer sequence, differing from another associated sequence by the order of some of the bases. In the path representation, this structural difference is represented by the permutation of the order of edges that form the two paths. For convenience and algorithmic effectiveness additional connections between the ends of biopolymer sequence (e.g. between 5′ and 3′ ends of DNA strand) can be added. The sequence-representing path of such a modified sequence graph is a cycle (which can be later opened at different sequence positions to restore the real molecular structure of the linear biopolymer. It was proven for the purposes of this patent disclosure that any cyclic path in any Eulerian graph could be decomposed into a unique finite set of subgraphs that we call basic cycles of a sequence graph. This proof is based on the fact that all basic cycles are balanced, oriented graphs (the number of in-edges and out-edges is the same—1 in and 1 out or 0 in and 0 out—for every vertex in any basic cycle). The union of any number of cycles into a sequence graph will thus necessarily generate a balanced, oriented graph. It is known that every balanced oriented graph is Eulerian and thus represents some biopolymer sequence.

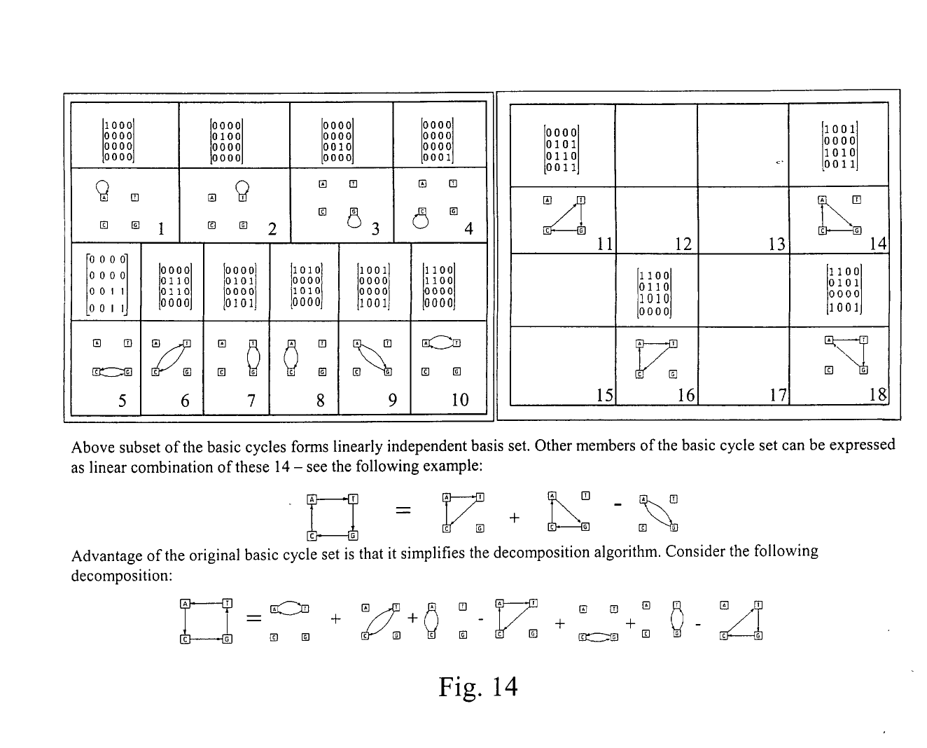

For 4-vertex sequence graphs representing DNA sequences, there are only 24 basic cycles, which are shown in FIG. 9. FIG. 10 depicts the decomposition of a sequence graph into these basic cycles. There are no other basic cycles that can represent DNA sequence other than those shown in FIG. 9. Thus, all other cycles (subcycles) in a DNA sequence graph are necessarily linear combinations of these 24 basic cycles. In fact, there are only fourteen linearly independent basic cycles, so all sequence graphs are necessarily linear combinations of the 14 linearly independent basic cycles (which are shown in FIG. 14). For the purposes of this invention, it is acceptable (although not preferable) to define sequences in terms of the 14 linearly independent basic cycles.

Thermodynamically, the content of particular basic cycles in any given sequence graph can be related to the number of nearest neighbor (loops and 2-cycles), next nearest neighbor (3-cycles) and next-next nearest neighbor (4-cycles) interactions of the bases in the primary structure of the described DNA. Matrix Representation of the Basic Cycles of Sequence Graphs As each of the basic cycles is a Eulerian graph, an adjacency matrix can represent them. Adjacency matrices that represent the basic cycles have only 0 and 1 elements (see FIG. 9). This property of matrix representation of basic cycles proves the uniqueness of exactly 24 basic cycles for DNA sequence graph decomposition. (As there is mathematical equivalence between any sequence graph and its adjacency matrix representation, which is an n×n matrix of positive integer numbers, the integer number elements of the adjacency matrix are necessarily sums of ones. To ensure that the sums of ones used as the elements of an n×n matrix form an adjacency matrix of an Eulerian sequence graph, the elements should be placed topologically correctly in the matrix so as to ensure that the resulting sums will represent matrix elements obeying the restrictions of a matrix generated from a Eulerian graph. This can be ensured only for the 24 basic cycle matrices shown in FIG. 9. The number of basic cycles of each type (e.g., for sequence graphs of DNA molecules, the number of basic loops, 2-cycles, 3-cycles and 4-cycles) is given by the number of ways the necessary 0-1 topologies can be generated in the n×n matrix. Matrix representations of the basic cycles are instrumental for the implementation of computer algorithm for decomposing sequence matrix into the basic cycles.

Compact Representation of the Basic Cycles of Sequence Graphs

For the purposes of optimizing algorithms, e.g. the SEQ-TG™ algorithm, and their implementation in the software code, another novel, non-obvious, more compact and memory-saving digital representation of the basic cycles was developed for DNA sequence graphs. Matrix representation of each basic cycle sequence graph requires 4×4=16 numbers with memory overhead for array indices. Nevertheless, the information that needs to be stored is only the relative or absolute chemical identity of vertices and the topology of up to four edges. Given the number of basic cycles for DNA (24), each cycle can be identified by using three digital labels: 0,1 and-1. One possible scheme is shown in Table 1. Up to number 18, the translation is straightforward: positive values indicate a move to the right, negative values a move to the left; start at the most left and return to the same place to finish the cycle.

TABLE 1

| Cycle | A | T | C | G |
|---|---|---|---|---|
| 1 | 1 | 0 | 0 | 0 |
| 2 | 0 | 1 | 0 | 0 |
| 3 | 0 | 0 | 0 | 1 |
| 4 | 0 | 0 | 1 | 0 |
| 5 | 0 | 0 | 1 | 1 |
| 6 | 0 | 1 | 1 | 0 |
| 7 | 0 | 1 | 0 | 1 |
| 8 | 1 | 0 | 1 | 0 |
| 9 | 1 | 0 | 0 | 1 |
| 10 | 1 | 1 | 0 | 0 |
| 11 | 0 | 1 | −1 | 1 |
| 12 | 0 | 1 | 1 | 1 |
| 13 | 1 | 0 | 1 | 1 |
| 14 | 1 | 0 | −1 | 1 |
| 15 | 1 | −1 | 1 | 0 |
| 16 | 1 | 1 | 1 | 1 |
| 17 | 1 | −1 | 0 | 1 |
| 18 | 1 | 1 | 0 | 1 |
| 19 | 1 | −1 | 1 | −1 |
| 20 | 1 | 1 | −1 | 1 |
| 21 | 1 | −1 | −1 | 1 |
| 22 | 1 | 1 | 1 | 1 |
| 23 | 1 | −1 | 1 | 1 |
| 24 | −1 | −1 | 1 | 1 |

Cycle Coefficient Vectors

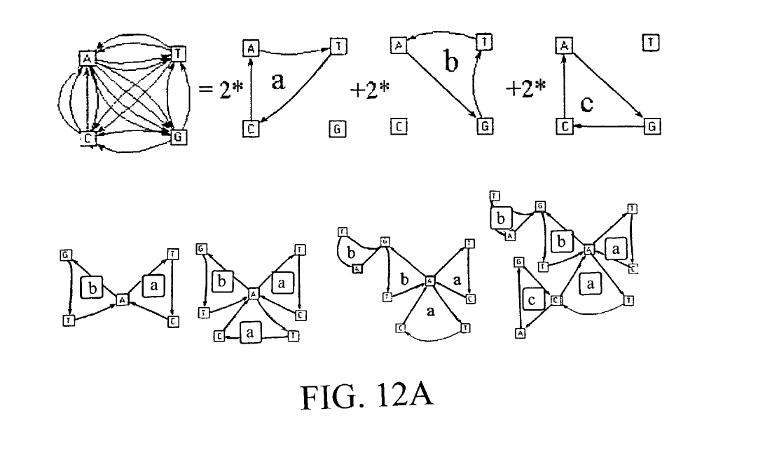

Another novel and non-obvious representation of nucleic acid sequence, such as DNA, that represents an isothermal set of sequences and is very efficient for database representation of sequence context and thermodynamics is a 24-element vector, the cycle coefficient vector, indicating of the number of basic cycles that are needed to decompose a particular sequence graph. FIG. 11 depicts the generation of a cycle coefficient vector for a particular DNA sequence graph. Indices associated with the basic cycles are chosen as the indices of the elements of the cycle coefficient vector. The numbers of basic cycles of a given type are entered as elements with corresponding indices and form the particular cycle coefficient vector. This representation of the sequence has several advantageous features. First, all sequences with identical (identity is defined by the general mathematical identity condition for vectors) cycle coefficient vectors will be isothermal. This follows from the fact that combining a specific set of basic cycles necessarily generates one and only one sequence graph. It was already shown that sequences incident to a particular sequence graph are predicted to be isothermal up to the nearest-neighbor approximation. Second, the cycle coefficient vector representation of the thermodynamic stability of a DNA sequence and its contextual topology has a structure of 24 vector elements that is independent of sequence length. Therefore, for example, normalization of cycle coefficient vector elements by the vector norm provides a descriptor capable of various systematic and quantitative comparisons of relative thermodynamic and topological properties of DNA sequences of vastly different lengths. Also the necessary context and stability information about biopolymer sequence with length approaching that of an organism's genome can be stored in single 24-element vector. Third, the cycle coefficient vector offers a convenient way to restrict input into sequence generating algorithms simply by setting the proper elements to zero (or any other pre-set value). For example, a sequence which obeys the condition that cycle coefficient vector element C1 is zero is guaranteed to contain no contiguous stretches of A bases.

Permutable Sequence Units

Novel and non-obvious implementation of permutable sequence units is necessary for SEQ-TG™ implementation that can be realized using real life computer hardware. Although the above-defined mathematical descriptors provide for the generation of actual biopolymer sequences with desired properties, for sequences longer than about 30 to 35 monomer units the combinatorial complexity of algorithms necessary for sequence generation might overflow capacity of current or even future computers and storage media. It is therefore desirable and necessary to reduce this complexity.

The descriptor that ultimately provides actual sequences is the sequence graph. To accomplish sequence generation, all paths (cyclic paths) in any such graph should be found. The polymer primary sequence is then given by the order of vertex labels as visited along each path. There do exist algorithms to find these paths, which are primarily of theoretical mathematical significance although they might be realized in software packages. Their common feature is that every generation of sequence of length N consists of N and more algorithmic steps, because these algorithms cycle through all edges of the sequence graph in every sequence-generating step. For certain topologies of sequence graphs and sequence lengths over 30 monomer units, it is easy to have 1022 or more sequences incident with a given graph. Even super fast computer with unlimited memory cannot process such data in practical amount of time. On the other hand, because the primary application of SEQ-TG™ is to find DNA sequences for microarray applications that should be isothermal and non-cross hybridizing, most of sequences systematically generated from any given sequence graph will be rejected in further selection steps, because they will have an unacceptable degree of homology. Permutable sequence units are designed to minimize the combinatorial complexity of the sequence generation process and enable implementation of algorithmic conditions that eliminate molecularly unacceptable sequences as soon as they reach threshold homology, thus further dramatically reducing the time required for the sequence generation. Permutable sequence units will be introduced using the example of DNA sequence graphs. The generalization for another linear polymers is straightforward.

The design of permutable sequence units is based upon the ability to uniquely decomposition a sequence graph into basic cycles. Each basic cycle represents segment of the final path that cannot be further subdivided into smaller segments. Thus if the sequence graph decomposition contains a 4-cycle, this 4-cycle represents a DNA segment, say ˜ACTG˜, that should appear as such in all sequences incident with the corresponding sequence graph. Depending upon the topology of the sequence graph, there might be even longer sequence segments that should be unchanged in all paths through the parent sequence graph. To find these segments, we use the properties of basic cycles into which the sequence graph is decomposed. Any path in a sequence graph follows sub-paths defined by basic cycles. Joining the basic cycles from the set into which the sequence graph was decomposed by their commonly labeled cycle vertices creates a path that is present in the sequence graph. Generation of a path present is a sequence graph is thus transformed into a combinatorial graph operation that consists of joining basic cycles through identically labeled vertices.

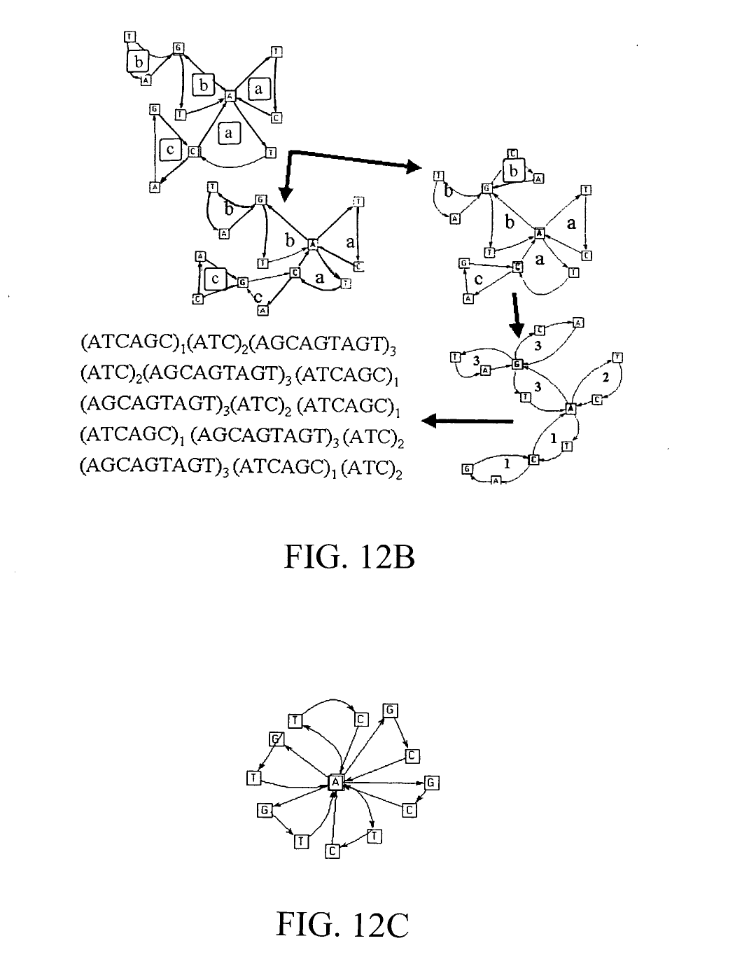

FIG. 12 depicts this process. FIG. 12A depicts a graph representing a 12-base DNA sequence is decomposed into three pairs of basic 3-cycles: a, b and c. In the next step, one a-cycle is joined through a commonly labeled vertex A with a b-cycle (these two basic cycles can also be joined via their T vertices, a possibility omitted in the scheme for clarity). Another a-cycle is then added to the subgraph. In this case, the a-cycle is connected via vertex A, one of four possible ways that it could have been joined (the other three include two through T vertices and one through C vertices, not shown). Next, the second b-cycle is added through common vertex G (again, all other possible topologies of adding this cycle are omitted). One of the possible additions of the first c-cycle is also shown.

In FIG. 12B, two of many possibilities are shown for how to add the last remaining c-cycle. The final graph that contains all basis cycles into which the original sequence graph was decomposed is then show in the right bottom part of the scheme. It is obvious that vertex A has unique topological role in the resulting graph. It is a nodal point of this particular path and thus forms the natural boundary between three sub-paths labeled 1, 2 and 3. Sub-paths starting at this vertex and following the oriented edges define three sequence units ATCAGC (unit 1), ATC (unit 2) and AGCAGTAGT (unit 3). The order of these units can be arbitrarily permuted and all new sequences resulting from these permutations are incident with the original sequence graph, which can be verified by converting them back into sequence graph.

The thermodynamic meaning of this result is as follows: for any cyclic nucleic acid sequence, the algorithm described above defines monomer units (sequence segments) that will have the same stability up to the nearest-neighbor energetic contribution, irrespective of their relative position(s) in the nucleic acid sequence. In other words, any permutation of these segments preserves the number and character of stacking interactions throughout the cyclic sequence.

FIG. 12C depicts an example of an alternative assembly of the same basic cycles shown in FIG. 12A. In this particular realization, all of the cycles are joined through a common vertex A. This example is a special case in which the permutable sequence units are all of equal length (ATC, AGC, and AGT—these correspond directly to basic cycles a, b and c) and the units can be placed in any order to form 12-mers that are isothermal.

Differences in Tₘ for sequences synthesized from these units will be due to end-effects (missing stacking where the cycle is open), as well as next nearest-neighbor and higher stability terms.

The process in which all possible combinations of basic cycle joining are determined is computationally extensive. For software implementation of the complete process compact encoding of the basic cycles is employed.

The Sequence Turbo Generator (SEQ-TG™)

An embodiment of the present invention is the Sequence Design Turbo Generator, SEQ-TG™ (Bioinformatics DNA Codes, Chicago, Ill.). Compared to present practices known in the art, SEQ-TG™ provides, among other things, optimal sequence design and generation of sequences for oligomer based applications, e.g., nucleic acid diagnostic microarrays.

The SEQ-TG™ technology is enabled by a novel functional representation of DNA sequence, the CFD. The CFD that explicitly depends on sequence context, as described above, is the basis of the SEQ-TG™ technology. The SEQ-TGTM is an analytical process comprised of computer driven algorithms that utilize specified sequence dependent input parameters and user defined sequence constraints. It provides for de novo design of sets of nucleic acid oligomer sequences with precisely defined properties, and selection of subsets of sequences from larger sequence sets that have the desired predicted properties. The SEQ-TG™ can be applied to generate sequences with optimum multiplex compatibility for use on microarrays or in multiplex solution applications, or for purposes of designing optimal and unique probes and primers. At the most basic level the entire process is founded upon comparisons of perfect duplexes and comparisons of imperfect duplexes (containing mismatches or internal loops) with perfect duplexes.

The disclosures of all of the publications (including patents and articles) cited herein are fully incorporated herein by reference.

From the foregoing, it will be observed that numerous modifications and variations can be effected without departing from the true spirit and scope of the present invention. It is to be understood that no limitation with respect to the specific examples presented is intended or should be inferred. The disclosure is intended to cover by the appended claims modifications as fall within the scope of the claims.

### EXAMPLES

### Example 1

### Single Base Mismatch Hybridization

In this and other examples of the invention, nucleic acid samples are prepared and melting curves collected following the procedures described in Owczarzy, R., et al. Biopolymers 44:217-239 (1997); Owczarzy, R., et al. Biopolymers 52:29-56 (2000). Synthetic DNA strands are typically purchased from commercial suppliers and purified according to established procedures. Doktycz, M. J., et al. Biopolymers, 32:849-864 (1992); Owczarzy, R., et al. Biopolymers 44:217-239 (1997); Benight, A. S., et al. Adv. Biophys. Chem., 5:1-55 (1995).

In this example of single base mismatch hybridization, SEQ-TG™ was employed to analyze the observed difference in stability of two 31 base pair duplex DNA molecules containing the same single base pair mismatch (A/C), flanked by the same nearest-neighbor base pairs on both the 5′ side (A−T) and 3′ side (G−C), but having the mismatched sequence (AAG/TCC) present at different positions within the duplex.

The PM (perfect match) duplex is composed of SEQ ID NO:1 and SEQ ID NO:2, where all of the bases between the two strands are the standard AT and GC base pairs.

SEQ ID NO:1 5′-taa aag ata cca tca atg agg aag ctg cag a-3′

SEQ ID NO:2 3′-att ttc tat ggt agt tac tcc ttc gac gtc t-5′

The MM (L) (mismatch left) duplex has a mismatch at the underlined position when the two oligomers are optimally aligned.

SEQ ID NO:1 5′-taa aag ata cca tca atg agg aag ctg cag a-3′

SEQ ID NO:3 3′-att tcc tat ggt agt tac tcc ttc gac gtc t-5′

The MM (R) (mismatch right) duplex has a mismatch at the underlined position when the two oligomers are optimally aligned.

SEQ ID NO:1 5′-taa aag ata cca tca atg agg aag ctg cag a-3′

SEQ ID NO:4 3′-att ttc tat ggt agt tac tcc tcc gac gtc t-5′

At these different positions the mismatch resides in different sequence contexts and the influence of sequence context on the thermodynamics of the A/C mismatch can be assessed. The analysis leads to a correction factor for calculating stability of an AAG/TCC mismatch that depends on sequence context.

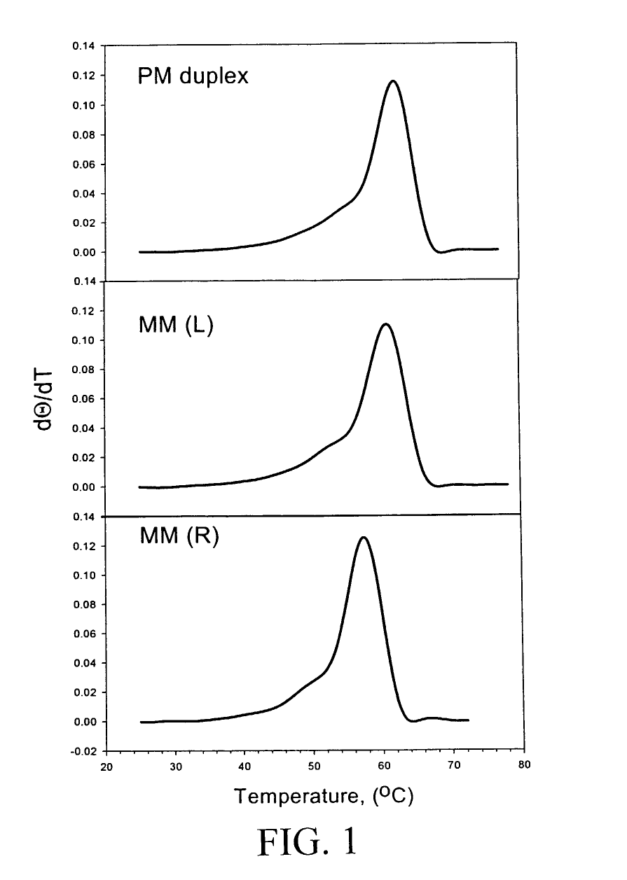

The differential melting curves obtained from absorbance versus temperature measurements on the duplexes, PM, MM (L), and MM (R), under identical solvent conditions (0.055 mM Na+) and at the same strand concentration (2.6 μM) are shown in FIG. 1. The measured tₘ(EXP) for each duplex obtained from the optical melting curves is given in Table 2, below, along with predictions made for the sequences using HyTher™, a published conventional method (Owczarzy et al).

TABLE 2

| | tₘ | tₘ | tₘ | |
|---|---|---|---|---|
| Duplex | (obs.) | Convent. | HyTher ™ | Q (cal/mol) |
| PM | 62.0° C. | 63.9° C. | 62.4° C. | 0.0 |
| MM (L) | 60.9° C. | 54.8° C. | 58.3° C. | 40300 |
| MM (R) | 57.4° C. | 54.6° C. | 58.3° C. | 43500 |

TABLE 3

| Duplex | Δtₘ (obs.) | Δtₘ Convent. | Δtₘ HyTher ™ |
|---|---|---|---|
| PM | 0° C. | 0° C. | 0° C. |
| MM (L) | −1.1° C. | −9.1° C. | −4.1° C. |
| MM (R) | −4.6° C. | −9.3° C. | −4.1° C. |

The data reveal that the tₘ's of the mismatched duplexes are less than the perfect match, but not by the same amount. As shown in Table 3, Δtₘ for the perfect match (PM) is 62.0° C., while tₘ is 60.9° C. for the duplex with the mismatch on the left side (MM(L)) and 57.4° C. for the duplex with the mismatch on the right side (MM(R)). The predicted tₘ value from HyTher™ is in good agreement for the PM duplex (62.4 versus 62.0° C.) but not in as good agreement for the mismatches.

The Δtₘ is the observed difference in melting temperatures, i.e. the melting temperature of the mismatch (MM) less the melting temperature of the perfect match (PM), as observed (obs.), as predicted by the conventional method (Convent.), and as predicted by HyTher™, respectively.

Not surprising, HyTher™ predicts tₘ of the mismatch duplexes to be the same. This would be expected because the calculation is based on the conventional nearest-neighbor model. Since identity of the mismatch and flanking nearest-neighbor base pairs are the same, the calculation predicts the same tₘ. In contrast, experimental results show that the tₘ of the 31 base pair duplexes containing a single base pair mismatch depends on the sequence context of the mismatch.

SEQ-TG™ analysis was employed to evaluate the correction factor required to account for the effects of sequence context on predicted tₘ. The following steps were part of the process:

(1) a CFD was constructed for each duplex sequence. FIG. 2 shows a schematic of some of the alignment positions examined to construct the CFD. The corresponding CFD's expressed in terms of the calculated tₘ, are shown in FIG. 3.

(2) the CFD of each mismatch was compared quantitatively with the CFD of the perfect match and the variance, Q, between the CFD of each mismatched duplex and the perfect match duplex was calculated.

(3) a direct relationship between the tₘ of the reference molecule (PM), tₘ(PM), and the tₘ's of the mismatches, tₘ(MM), was identified:

tₘ(PM)=tₘ(MM)·(Q)·(k)

where k is the “context correction factor” and equals 2.0×10⁻⁵. The context correction factor carries, through Q, the overall differences in the context of mismatches as compared to the perfect match. The variance between the reference and mismatched CFD's is directly proportional to the context dependent correction factor.

The Q value given in the far right column of Table 2 is lower for the mismatched duplex MM(L), indicating that MM(L) is relatively more stable then MM(R) and thus closer to the stability of the PM duplex. This means that the greater the difference in tₘ, Δtₘ, between a mismatched and perfect match duplex, the larger the variance between their CFD's.

### Example 2

### Cross-Hybridization

For the cross-hybridization example, SEQ-TG™ was applied to analyze groups of sequences with a propensity for cross-hybridization. Cross-hybridization results from hybrid duplex states other than the perfectly matched duplex that can form when single strands from several different perfect matched duplexes are simultaneously present in solution. Obviously, such “side-reactions” are of particular nuisance in multiplex reactions because they result in lower accuracy, precision and sensitivity of microarray data and its interpretation. Effective sequence design minimizes likelihood for such reactions and allows for higher quality data.

For this example, eight pairs of strands were studied by optical melting analysis. The controls, or reference duplexes, were the four 24 base pair duplexes having the sequences shown below.

I_{P}(SEQ ID NO:5) 5′-gtt atg att gta gat aaa agg att-3′

I_{T} (SEQ ID NO:6) 5′-aat cct ttt atc tac aat cat aac-3′

II_{P} (SEQ ID NO:7) 5′-aag aga ttg tat tgt aga taa aag-3′

II_{T} (SEQ ID NO:8) 5′-ctt tta tat aca ata caa tat ctt-3′

III_{P}(SEQ ID NO:9) 5′-gtt aga ttt gat gta ttg tat tga-3′

III_{T} (SEQ ID NO:10) 5′-tca ata caa tac atc aaa tct aac-3′

IV_{P} (SEQ ID NO:11) 5′-ttg agt atg att tgt atg ata gaa-3′

IV_{T} (SEQ ID NO:12) 5′-ttc tat cat aca aat cat act caa-3′

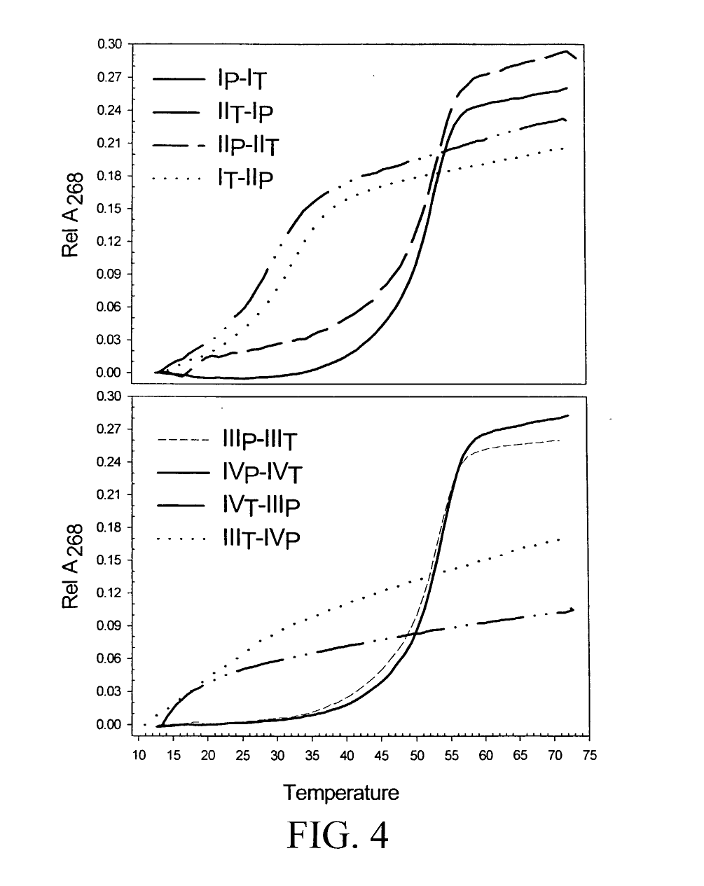

Each duplex is comprised of a probe (P) and target (T) strand having the designation I_{P}-I_{T}, II_{P}-II_{T}, III_{P}-III_{T}, or IV_{P}-IV_{T}. Melting curves, plots of the relative 268 nm absorbance increase as a function of temperature for the four duplexes alone in solution are shown in FIG. 4. Values of the experimentally determined melting temperatures, tₘ, obtained from each curve, and calculated tₘ's derived using the conventional methods and HyTher™ are shown in the Table 4.

TABLE 4

| | tₘ, ° C. | tₘ, ° C. | tₘ, ° C. |
|---|---|---|---|
| Duplex | Observed | Conventional | HyTher ™ |
| I_{T}-I_{P} | 52.7 | 53.4 | 51.0 |
| II_{T}-II_{P} | 52.7 | 52.8 | 50.6 |
| I_{T}-II_{P} | 29.2 | 26.4 | 36.5 |
| II_{T}-I_{P} | 32.5 | 34.5 | 36.1 |
| III_{T}-III_{P} | 51.7 | 54.8 | 51.2 |
| IV_{T}-IV_{P} | 52.6 | 54.3 | 51.6 |
| III_{T}-IV_{P} | ˜17 | 24.4 | 2.3 |
| IV_{T}-III_{P} | ˜15 | −14.7 | −32.4 |

In addition, the following hybrid strand combinations, I_{P}-II_{T}, II_{P}-I_{T}, III_{P}-IV_{T} and IV_{P}-III_{T}, were prepared and their melting curves measured. The first two pairs are possible combinations if duplexes I_{P}-I_{T} and II_{P}-II_{T} were to be both present in the same hybridization mixture. The latter two pairs are possibilities when III_{P}-III_{T} and IV_{P}-IV_{T} are both present in the same hybridization mixture.

The collected melting curves are shown in FIG. 4. Hyperchromicity changes for the four perfect match duplexes are from 22 to 29%, consistent with what might be expected for melting of 24 base pair duplexes. Hyperchromicity changes for the hybrid mixtures were only slightly less for the IP-II_{T} and II_{P}-I_{T} mixtures (18 to 22%) but significantly lower for the III_{P}-IV_{T} and IV_{P}-III_{T} mixtures (9 to 15%). Albeit relatively less than observed on melting curves of the perfect match duplexes, these hyperchromicity changes for the hybrid mixtures reveal some amount of complex formation.

The tₘ's estimated from these hybrid melting curves given in the above table, are considerably lower than for the perfect matched duplexes. Independent confirmation of complex formation for the III_{P}-IV_{T} mixture was also obtained from differential scanning calorimetry measurements (not shown).

To ascertain whether the examination of these mixtures alone was representative of the situation where both perfect duplexes are present, pairs of duplexes, perfect duplex plus hybrid mixture were also melted. On these melting curves two transitions, presumably corresponding to the perfect duplex and hybrid structure(s), were observed (not shown).

Apparently, the hybrid duplex forms in the presence of the perfect match duplex.

Calculated values of tₘ for the perfect duplexes and various hybrid mixtures from the HyTher™ program are given in the table above. Calculations for the perfect match duplexes were straight forward. For the hybrid mixtures, structures of potential complexes had to be assumed. For these calculations each pair of sequences were input into the primer walk option of the HyTher™ program and the most stable alignment, and corresponding tₘ of that alignment, were determined. Comparisons of the calculated and experimental results in the table above reveal tₘ values for the perfect duplexes are reasonably predicted by HyTher™ (within 2.8° C.). For the hybrid mixtures, II_{P}-I_{T} and I_{P}-II_{T}, predicted tₘ values for the most likely alignments are 7.3 and 3.6° C. higher, respectively, than observed experimentally. These predictions suggest the hybrid mixtures would probably cross-hybridize with sufficient stability. For the IV_{P}-III_{T} and III_{P}-IV_{T} mixtures tₘ predicts tₘ values approximately 15 and 47° C., respectively, lower than observed experimentally! In contrast to the other strand mixtures, these predicted tₘ values are so low compared to the perfect duplexes that these mixtures would be predicted not to cross-hybridize. Experimentally, these hybrid duplexes are much more stable than predicted by HyTher™.

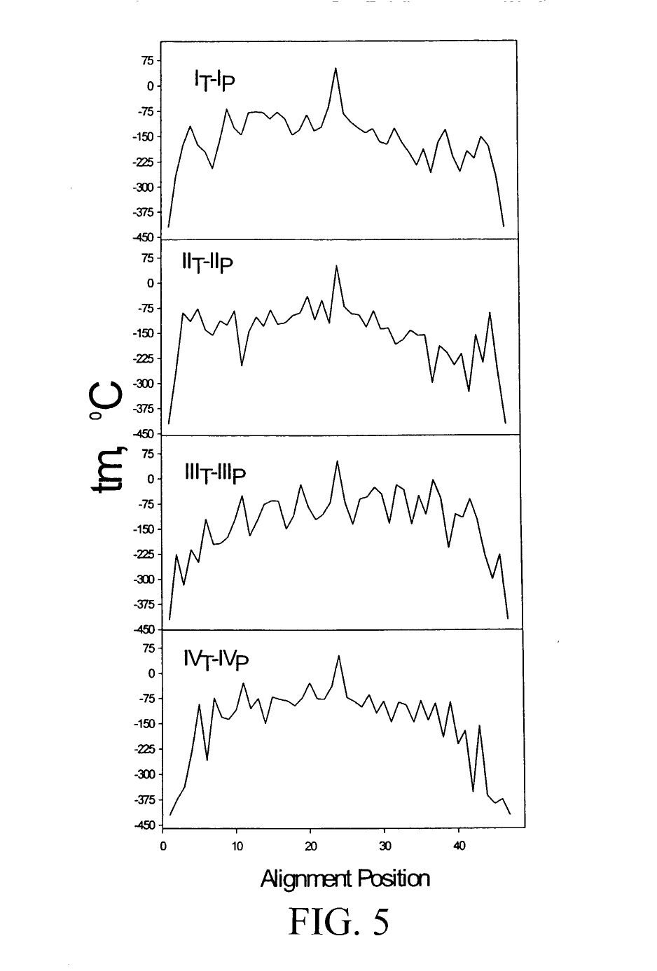

The above result reveals a shortcoming of the conventional approach, so we applied the SEQ-TG™ to gain some insight into the observed, but not anticipated cross-hybridization behavior. Through this process a new source of sequence dependent thermodynamic stability in hybrid duplexes was divulged. Following are steps in the analytical procedure that was employed. (1) The CFD of each pair of strands that were melted, the perfect duplexes and hybrid mixtures were constructed in two different ways. Initially, each CFD was constructed using published values of the hydrogen bonding contribution (A−T or G−C), nearest-neighbor, next nearest-neighbor stacking interactions published by Benight and coworkers and the nearest-neighbor dependent single base pair mismatch values published by SantaLucia and coworkers. See Benight, A. S., et al. Adv. Biophys. Chem., 5:1-55 (1995); and SantaLucia J. Jr., “A unified view of polymer, dumbbell, and oligonucleotide DNA nearest-neighbor thermodynamics,” Proc. Nat'l Acad. Sci., U S A., 95:1460-1465 (1998). Benight's values for the nearest-neighbor sequence dependent calculations were used here since they have been shown several times to be comparable to SantaLucia's and would not be expected to yield incomparable results. For each alignment the quantitative nearest-neighbor parameters were used to predict the respective tₘ. The CFD's for the perfect match duplexes are shown in FIG. 5. On the CFD's for the perfect match duplexes, a maximum occurs at the perfect alignment position that is in every case within 3° C. of the experimental tₘ. This is comparable to the predictions obtained for the perfect matched duplexes with HyTher™, and supports the assertion that the Benight and SantaLucia nearest-neighbor sets produce comparable predictions.

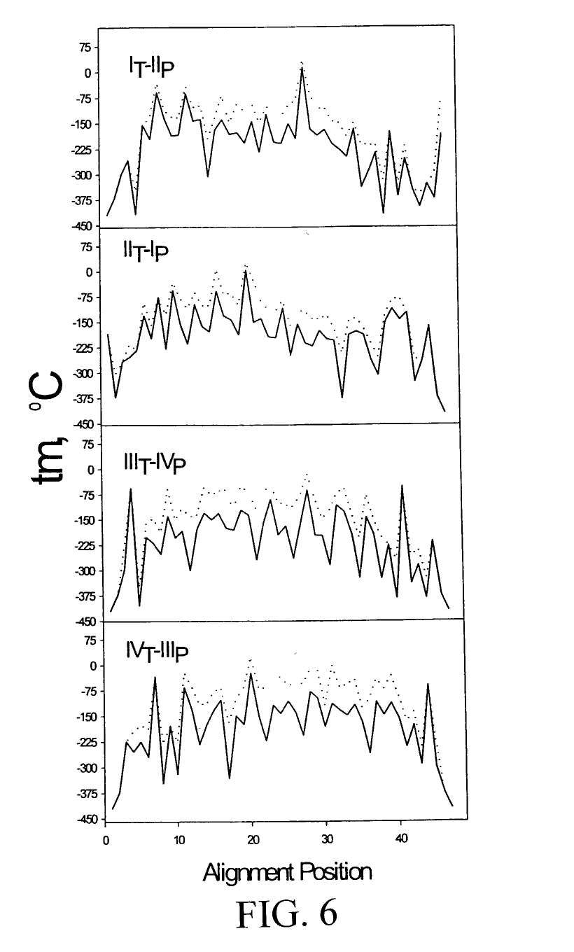

For the hybrid mixtures, sequences were aligned in the various possible configurations and for each of these the tₘ (and corresponding thermodynamic transition parameters) were calculated by counting the number of complementary base pairs, nearest neighbor stacking interactions and base pair mismatches present at each alignment position. This process was continued until the state with the maximum number of base pairs and highest predicted tₘ was formed, corresponding to a maximum on the CFD, with height corresponding to the calculated tₘ. The CFD's for the hybrid mixtures are shown in FIG. 6 (solid lines). When the standard prescription for calculating stability is followed, i.e. considering only hydrogen bonding contributions (A−T or G−C), nearest-neighbor, next nearest-neighbor stacking interactions and nearest-neighbor dependent single base pair mismatches in hybrid duplex states to calculate tₘ, the conventional method produced calculated tₘ's (not shown) quite comparable to those predicted by HyTher™.

A tenet of the present invention is that the degree of similarity between the CFD of the hybrid mixtures and the CFD of the reference (perfect matched duplex) is a predictor of the propensity for complex formation (cross-hybridization) in the hybrid mixtures. Practically, this depends of course on the quantitative features and quality of the CFD's constructed for the hybrid duplexes. Results of the quantitative comparisons of the various hybrid CFD's with those of corresponding perfect match duplexes are summarized in the table above.

When the CFD's are calculated in the conventional manner, correlation coefficients range from 0.47 to 0.65. Since experimental tₘ's were much higher than predicted by the standard method using HyTher™ or the Benight parameters, additional stabilizing interactions not considered in the standard method must be included.

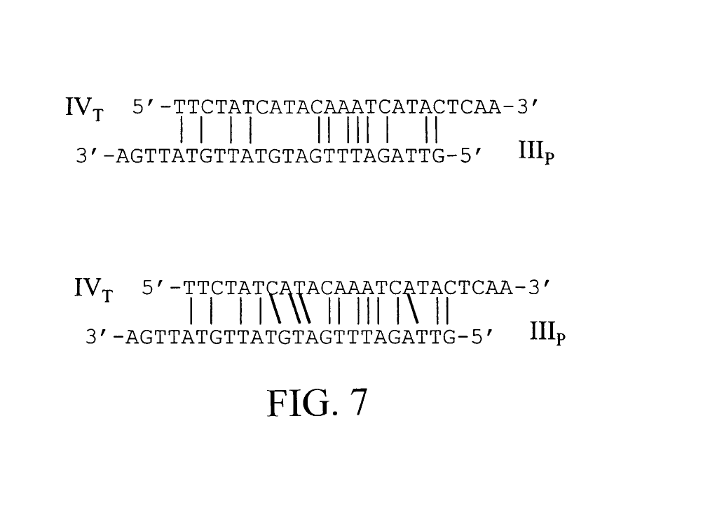

The hybrid mixtures IV_{P}-III_{T} and III_{P}-IV_{T} show the greatest departure from the conventional predictions, so for example, the following discussion focuses on the III_{P}-IV_{T} hybrid mixture. For this pair of strands the standard alignment procedure was performed and the specific alignment that produced the most hydrogen bonds between complementary base pairs was denoted. This aligned state is shown in FIG. 7. Examination of this state and its sequence suggested immediately a possible source of the observed much higher than expected stability. That is, hydrogen bonding of complementary bases within internal loops comprised of two more adjacent base pair mismatches. To test this hypothesis, the conventional calculation was augmented to consider this source of added stability. Specifically, where adjacent mismatches occurred and bases in adjacent positions on opposite strands that were complementary occurred, a stabilizing factor equal to a fraction (0.6) of the total hydrogen bonding stability of a base pair was assigned to each occurrence. As shown in FIG. 7 (bottom figure), there are four such additional interactions (depicted by dark bars) that might contribute added stability to the hybrid duplex complex.

With this additional thermodynamic contribution a new set of CFD's was constructed for the hybrid duplexes using the SEQ-TG™. These plots are shown in FIG. 6 (dotted lines) and compared directly to the CFD's constructed in the conventional way (solid lines). The tₘ's obtained using these new CFD's for the hybrid mixtures are summarized in the above table (SEQ-TG™), and are in much better agreement with experimental measurements. Again, the newly constructed CFD's of the hybrid mixtures were quantitatively compared with the CFD's of the corresponding perfect match duplexes. The resulting correlation coefficients are summarized in the table below under the column SEQ-TG™. As can be seen these values increase dramatically when the CFD's are constructed considering the suggested intraloop stabilizing interactions. This means that the newly calculated CFD's of the hybrid duplexes are much more similar to the perfect match, and now, consistent with experimental observations, would be expected to have a higher propensity of cross-hybridization.

TABLE 5

| CFD Profile Similarity | | |
|---|---|---|
| Correlation Coefficients, R | SEQ-TG ™ | SEQ-TG ™ |
| Duplex Profile Comparison | (Conventional) | (Optimized) |
| I_{T}-I_{P} vs. II_{T}-I_{P} | 0.54871 | 0.82672 |
| I_{T}-I_{P} vs. I_{T}-II_{P} | 0.50019 | 0.80676 |
| II_{T}-II_{P} vs. I_{T}-II_{P} | 0.54542 | 0.83762 |
| II_{T}-II_{P} vs. I_{T}-II_{P} | 0.47175 | 0.806711 |
| III_{T}-III_{P} vs. IV_{T}-III_{P} | 0.65258 | 0.90985 |
| III_{T}-III_{P} vs. III_{T}-IV_{P} | 0.60078 | 0.89388 |
| IV_{T}-IV_{P} vs. IV_{T}-III_{P} | 0.60592 | 0.91229 |
| IV_{T}-IV_{P} vs. III_{T}-IV_{P} | 0.53627 | 0.89523 |

There are two obvious, very important practical implications of these results. First, results suggest that thermodynamic stabilizing interactions might occur in internal loops comprised of more than two base pair mismatches. Second, when the SEQ-TG™ includes this new thermodynamic component, more quantitative estimates of cross-hybridization propensity can be obtained from the correlation coefficient A number of embodiments of the invention have been described. Nevertheless, it will be understood that various modifications may be made without departing from the spirit and scope of the invention. Accordingly, other embodiments are within the scope of the following claims.

What is claimed is:

1. A method of analyzing a nucleic acid sequence comprising:
constructing a CFD,
thereby analyzing a nucleic acid sequence.

2. A method of identifying a CFD component associated with a property of a nucleic acid sequence or a peptide encoded by the nucleic acid, comprising:
optionally, providing CFDs for a training set of nucleic acid sequences;
identifying one or more components of the CFDs;
identifying a component, the presence, value, or contribution of which, is correlated, negatively or positively, with a property of the nucleic acid or the peptide encoded by a nucleic acid,
thereby identifying a CFD component associated with a property of a nucleic acid sequence or a peptide encoded by the nucleic acid.

3. A method of analyzing a nucleic acid sequence, comprising:
providing a CFD for the nucleic acid sequence;
identifying one or more components of the CFD;
determing if a preselected component, known to be associated with a property of the nucleic acid sequence or a peptide encoded by the nucleic acid, is present,
thereby analyzing the nucleic acid sequence.

4. A method of comparing nucleic acid sequences, comprising:
representing a nucleic acid sequence by a mathematical function of the entire sequence context, that depends on the collective characteristics or attributes of sequence type, order and composition, (a CFD); and
comparing CFD's of two or more different, but perfectly matched, duplex sequences by providing a quantitative measurement of similarity between their CFDs.

5. The method of claim 4, wherein the method further includes comparing the CFD(s) of one (or more) hybrid duplexes comprised of two strands, whose sequences are not perfectly complementary, with the CFD(s) of the prefect duplexes comprised of one of each strand of the hybrid duplex and its perfect complementary strand.

6. The method of claim 5, wherein the method further the following steps:
calculating the CFD's for all duplexes under consideration;
recording the CFD for each pair of strands in each prefect duplex under consideration.

7. The method of claim 5, wherein the quantitative similarity of the shapes of the reference CFD's and CFD's constructed for pairs of strands from different perfect duplexes provides a quantitative indication of the propensity for cross hybridization of the imperfect matched strands, which is useful where various pairs of strands are simultaneously present in a solution as is the case in a multiplex environment.

8. The method of claim 5, wherein the method further includes predicting both the transition temperature and cross-hybridization of duplex sequences from the CFD, and includes the following steps:
providing a set of duplex DNA molecules;
providing the melting temperature of each duplex;
measuring the cross-hybridization behavior of the set of duplexes;
calculating the CFD's for the perfect duplex molecules of the set and of all the other combinations of strands and recording them, to provide a training set for an artificial intelligence algorithm;
simplifying the CFD input by finding the basis CFD's for the set which are the minimal number of CFD's that can be combined to produce the entire set of CFD's;
relating the coefficients of each sequence with the observed transition temperature and cross-hybridization propensity; and
predicting the transition temperature and cross hybridization propensity for any new sequence from the coefficients of the basis CFD's for that sequence.

9. The method of claim 5, wherein the method is applied to predict the shape of the CFD from the desired transition temperature and cross hybridization propensity comprised of the following steps:
providing preparing a set of duplex DNA molecules;
providing the melting temperature of each duplex;
determining the cross-hybridization behavior of the set of duplexes;
calculating the CFD's for the perfect duplex molecules of the set and of all the other combinations of strands and recording them to provide a training set for an artificial intelligence algorithm;
simplifying the CFD input by finding the basis CFD's for the set which are the minimal number of CFD's that can be combined to produce the entire set of CFD's. (For example, if three basis CFD's are found then the shape of the CFD for each pair of sequences can be represented by three numbers (coefficients) instead of an entire CFD);
training a neural network or using regression analysis to relate the observed transition temperature and cross-hybridization propensity with the coefficients representative of the CFD of each sequence; optimizing the neural network or regression by interactive adjustment using algorithms;
calculating the predicted CFD from the desired transition temperature and cross hybridization propensity;
feeding the desired Tₘ and cross-hybridization propensity into the trained network which provides the coefficients of the CFD; and
calculating the correponding CFD for the sequences with the desired Tₘ and cross-hybridization propensity.

11. The method of claim 5, wherein the method is applied to scanning of a nucleic acid, e.g., a gene or genome, and finding sequences with most similar and dissimilar segments and includes the following steps:
for analysis of a gene sequence (one strand) define the desired length, N, for a probe (primer or marker) to be compared to the gene sequence;
starting at the first base of the genome, calculate the CFD for the N base pair duplex from position 1 to position N, continuing the process moving over every N base pair sequence until the last n base pair duplex of the genome is considered; and
calculating the correlation coefficients for all combinations of perfect match duplex CFD's, recording the results as elements, rᵢⱼ, of a correlation matrix.

12. The method of claim 5, wherein the method determines the cross-hybridization propensity for a set of probes, e.g., all probes of a genome or a selected subset of dissimilar probes using a predefined threshold value of rij including the following steps;
provide all possible combinations of probe strands in duplexes;
provide the CFD's of all possible combinations;
after aligning each pair of CFD's at their minima, calculate the correlation coefficients of each pair of CFD's and assemble the correlation matrix.

13. The method of claim 5, wherein the method is used to scan a nucleic acid, e.g., a gene or genome sequence, for optimal regions for micro-array applications comprising the following steps.
define the Tₘ at which the micro array will be operated;
define the desired threshold for cross hybridization propensity;
define the length of the probes for the microarray;
using a trained neural network predict the coefficients of the basis CFD's from the desired Tₘ and cross-hybridization propensity;
use the basis CFD's and coefficients to generate the predicted CFD matching the desired Tₘ and cross-hybridization propensity; examine all sequences of the desired length and provide their CFD's;
determine quantitative similarity between calculated and predicted CFD'S;
label each position by its corresponding correlation coefficient;
define a threshold of similarity by the value of the correlation coefficient, for example rᵢⱼ>0.7.
thereby providing sections of the gene above this threshold and having the desired Tm and cross-hybridization propensity.

14. The method of claim 5, wherein the method is used to design and generate probe sequences for use in a universal sequence microarray comprising the following steps.
(a) generating an Eulerian graph, describing a plurality of nucleic acid sequences;
(b) partitioning the nucleic acid sequences according to a given composition;
(c) creating subgraphs that specify how many and what type of the monomeric basis comprise the sequences wherein the subgraphs have vertices that correspond to the types of oligomeric sequences and edges that correspond to partitioning of the integers that describe properties of the sequences;
(d) characterizing the sequences by their propensity for cross-hybridization by (i) formulating the context functional descriptor of each sequence aligned with itself as a nucleic acid duplex at each alignment position and (ii) assigning a number representing the relative thermodynamic stability of the duplex, thereby generating diagonal elements of a correlation matrix; and
(e) aligning the deepest minima of off-diagonal elements of the correlation matrix with the deepest minima of the diagonal elements of the correlation matrix, thereby analyzing the potential interactions between the nucleic acid sequences.

15. The method of claim 5, wherein the method analyzes the potential interactions between nucleic acid sequences, e.g., sequences described herein, wherein the subgraphs generated in step (c) are listed in a relative manner according a desired property.

16. A method for analyzing a population of nucleic acid sequences comprising:
providing a population of nucleic acid sequences;
providing a CFD for each nucleic acid sequence and each nucleic sequence of a selected group of complements of the nucleic acids of the population;
comparing the CFD for each nucleic acid sequence and its perfect complement with each of the CFD's for the same nucleic acid and each nucleic sequence of a selected group of complements of the nucleic acids of the population;
thereby analyzing a population of nucleic acid sequences, e.g., for selecting a subset of the population having a selected degree of cross-hybridization or non cross-hybridization.

17. The method of claim 16, wherein the calculation of CFD includes accounting for loop structures inferred from mismatches.

18. The method of claim 16, wherein the parameter can include one or more of a thermodynamic value.

19. The method of claim 16, wherein the comparing step can include aligning the CFD data by a selected characteristic of a curve of values from the CFD.

20. The method of claim 16, wherein the comparison can include calculating a matrix of n sequences, wherein the matrix is a, b, c×a′, b′, c′, and the values in the matrix represent the CFD for a given duplex.

21. A method of providing a population of nucleic acid sequences comprising:
a) providing a value for the length of a nucleic acid;
b) providing values for the base composition;
c) providing a Eulerian representation, of possible sequences which representation can be described by Eulerian graph,
d) extracting sequences from the representation,
to thereby provide a population of nucleic acid sequences.

22. The method of claim 21, wherein the Eulerian representation can be an n×n matrix, wherein n is equal to the number of bases used.

23. The method of claim 21 wherein extracting the sequence can include decomposing the Eulerian representation into components and permuting the components to produce the population of sequences.

24. A method of providing a population of nucleic acid sequences comprising:
a) providing a value for the length of a nucleic acid;
b) providing values for the base composition;
c) providing a representation, sometimes referred to herein as a Eulerian representation, of possible sequences which representation can be described by Eulerian graph;
d) repeating steps a, b, and c, at least one time;
e) extracting sequences from the representations,
to thereby provide a population of nucleic acid sequences.

25. The method of claim 24, wherein the representation can be an n×n matrix, wherein n is equal to the number of bases used.

26. A method for analyzing nucleic acid sequences comprising the steps of:
(a) generating an Eulerian graph, or representation thereof, describing a plurality of nucleic acid sequences;
(b) optionally, partitioning the nucleic acid sequences according to a given composition;
(c) creating subgraphs that specify how many and what type of the monomeric basis comprise the sequences wherein the subgraphs have vertices that correspond to the types of oligomeric sequences and edges that correspond to partitioning of the integers that describe properties of the sequences;
(d) characterizing the sequences by their propensity for cross-hybridization by (i) formulating the context functional descriptor of each sequence aligned with itself as a nucleic acid duplex at each alignment position and (ii) assigning a number representing the relative thermodynamic stability of the duplex, thereby generating diagonal elements of a correlation matrix;
(e) characterizing the sequences by their propensity for hybridization by (i) formulating the context functional descriptor of each sequence aligned with every other sequence as a nucleic acid duplex at each alignment position and (ii) assigning a number representing the relative thermodynamic stability of the duplex, thereby generating off-diagonal elements of the correlation matrix; and
(f) aligning the deepest minima of off-diagonal elements of the correlation matrix with the deepest minima of the diagonal elements of the correlation matrix, thereby analyzing the potential interactions between the nucleic acid sequences.

27. A method of and identifying a population of sequences comprising:
providing an initial population of nucleic acid sequences, e.g., cDNA's;
providing, for a first nucleic acid sequence of the population, a selected set of oligomers derived from the first nucleic acid;
providing, for a second and optionally subsequent nucleic acid sequence of the population, a selected set of oligomers derived from the second or subsequent nucleic acid;
providing a Tₘ, for oligomers produced above and its perfect compliment;
selecting subpopulations of the oligomers for which a Tₘ is provided into a plurality subpopulations each having a preselected range of values for Tₘ,
thus providing a subpopulation which has a selected property.

28. A method for analyzing a nucleic acid sequence, to determine the A Tₘ involved with introducing a change comprising:
providing a nucleic acid sequence A and providing a first CFD for the perfect duplex, A, A′;
providing a nucleic acid sequence B′ which is the complement of B and where B differs from A by a change;
providing a second CFD for the imperfect duplex, A, B′;
comparing the first and second CFD's,
providing a correlation coefficient
providing a value for Tₘ, for the perfect duplex A, A′;
determining a value for the parameter for the imperfect duplex A, B′ by dividing the Tₘ of A, B′ by the correlation coefficient, thereby analyzing a nucleic acid sequence.

29. The method of claim 28, wherein the change is a change at a single nucleotide giving a single nucleotide mismatch.

30. A computer readable file, having a record which includes an element which identifies a nucleic acid, and an element which describes the CFD or on or more components thereof.

31. The file of claim 30, wherein the record includes an element which identifies a property of the nucleic acid or the peptide it encodes.

32. The file of claim 30, wherein the file includes records for a plurality of nucleic acids.

33. A method of analyzing a nucleic acid sequence comprising: providing a Eulerian representation of a population of sequences, wherein the population includes at least 10⁵ sequences; searching the population for a sequence of interest or comparing a reference sequence with a sequence in the population.

34. A set of nucleic acids, made or compiled by a method described herein.

35. The set of nucleic acids of claim 34, wherein it is an ordered array.
